(19) United States
(12) Patent Application Publication
(10) Pub. No.: US 2026/0334926 A1
(43) Pub. Date: Oct. 29, 2026

(54) EXPANDABLE TISSUE ENGAGEMENT APPARATUS AND METHOD

(71) Applicant: Teleflex Life Sciences LLC, Wilmington, DE (US)

(72) Inventors: Curtis Yarra, Dublin, CA (US); Ernest Cabreza, Dublin, CA (US); Robert DeCou, Pleasanton, CA (US); Manoj Kumar Singh, Pleasanton, CA (US); Erik Noel, San Ramon, CA (US); Robert J. McCaffrey, Hillsborough, NH (US); Robert Lane, Milford, NH (US); David Michael Fournet, Nashua, NH (US); Derek John Hugger, Mont Vernon, NH (US); Joseph O. St. Cyr, Salem, NH (US); Byron Liehwah Chun, Castro Valley, CA (US)

(73) Assignee: Teleflex Life Sciences LLC, Wilmington, DE (US)

(21) Appl. No.: 19/657,688

(22) Filed: Apr. 24, 2026

Related U.S. Application Data
(60) Provisional application No. 63/794,653, filed on Apr. 25, 2025.

(51) Int. Cl. A61B 17/00

(52) U.S. Cl. CPC A61B 17/00234; A61B 2017/00274; A61B 2017/00367; A61B 2017/00477

(57) ABSTRACT

A system configured to engage one or more lobes of a prostate gland includes an implant delivery device including a handle assembly and an elongate shaft assembly attached to the handle assembly and configured for insertion into the urethra of a subject. The system also includes a tissue engagement structure configured to be attached to and removed from a distal end of the elongate shaft assembly. The system also includes a handheld loading tool configured to attach the tissue engagement structure to the distal end of the elongate shaft assembly and subsequently remove the tissue engagement structure from the distal end of the elongate shaft assembly in response to manual engagement.

### CROSS REFERENCE TO RELATED APPLICATIONS

This application claims priority to U.S. Provisional Patent Application No. 63/794,653, filed on Apr. 25, 2025 and titled “Expandable Tissue Engagement Apparatus and Method,” the entire contents of which are hereby incorporated by reference herein.

### TECHNICAL FIELD

The present disclosure relates generally to medical devices and methods, and more particularly to devices, systems, and associated methods for manipulating or engaging tissues and anatomical or other structures within the body of human or animal subjects for the purpose of treating diseases or disorders.

### BACKGROUND

One example of a condition where it is desirable to lift, compress, or otherwise remove a pathologically enlarged tissue is Benign Prostatic Hyperplasia (BPH). BPH is one of the most common medical conditions that affect men, especially elderly men. It has been reported that, in the United States, more than half of all men have histopathologic evidence of BPH by age 60 and, by age 85, approximately 9 out of 10 men suffer from the condition. Moreover, the incidence and prevalence of BPH are expected to increase as the average age of the population in developed countries increases.

The prostate gland enlarges throughout a man's life. In some men, the prostatic capsule around the prostate gland may prevent the prostate gland from enlarging further. This causes the inner region of the prostate gland to squeeze the urethra. This pressure on the urethra increases resistance to urine flow through the region of the urethra enclosed by the prostate. Thus, the urinary bladder has to exert more pressure to force urine through the increased resistance of the urethra. Chronic over-exertion causes the muscular walls of the urinary bladder to remodel and become stiffer. This combination of increased urethral resistance to urine flow and hypertrophy and stiffness of urinary bladder walls leads to a variety of lower urinary tract symptoms (LUTS) that may severely reduce the patient's quality of life. These symptoms include weak or intermittent urine flow while urinating, straining when urinating, hesitation before urine flow starts, feeling that the bladder has not emptied completely even after urination, dribbling at the end of urination or leakage afterward, increased frequency of urination particularly at night, and urgent need to urinate.

In addition to being present in patients with BPH, LUTS may also be present in patients with prostate cancer, prostate infections, and chronic use of certain medications (e.g. ephedrine, pseudoephedrine, phenylpropanolamine, and antihistamines such as diphenhydramine or chlorpheniramine) that cause urinary retention, especially in men with prostate enlargement.

Although BPH is rarely life threatening, it can lead to numerous clinical conditions including urinary retention, renal insufficiency, recurrent urinary tract infection, incontinence, hematuria, and bladder stones.

In developed countries, a large percentage of the patient population undergoes treatment for BPH symptoms. It has been estimated that by the age of 80 years, approximately 25% of the male population of the United States will have undergone some form of BPH treatment. At present, the available treatment options for BPH include watchful waiting, medications (phytotherapy and prescription medications), surgery, and minimally invasive procedures.

For patients who choose the watchful waiting option, no immediate treatment is provided to the patient, but the patient undergoes regular exams to monitor progression of the disease. This is usually done on patients who have minimal symptoms that are not particularly bothersome.

Medical procedures for treating BPH symptoms include Transurethal Resection of the Prostate (TURP), Transurethral Electrovaporization of the Prostate (TVP), Transurethral Incision of the Prostate (TUIP), Laser Prostatectomy, Open Prostatectomy, Transurethral Microwave Thermotherapy (TUMT), Transurethral Needle Ablation (TUNA), Interstitial Laser Coagulation (ILC), and Prostatic Stents.

The most effective current methods of treating BPH in terms of relieving the symptoms of BPH also carry a high risk of adverse effects. These methods may require general or spinal anesthesia and/or may have potential adverse effects that dictate that the procedures be performed in a surgical operating room, followed by a hospital stay for the patient. The methods of treating BPH that carry a lower risk of adverse effects are also associated with a lower reduction in the symptom score. While several of these procedures can be conducted with local analgesia in an office setting, the patient does not experience immediate relief and often experiences worse symptoms for weeks after the procedure until the body begins to heal. Additionally, many surgical or minimally invasive approaches require a urethral catheter to be placed in the bladder, and in some cases left in the bladder for weeks. In some cases, catheterization is indicated because the therapy actually causes obstruction during a period of time post operatively, and in other cases it is indicated because of post-operative bleeding and the potential for the formation of occlusive clots. While drug therapies are easy to administer, the results are frequently suboptimal, take significant time to take effect, and often include undesirable side effects.

There have been advances in developing minimally invasive devices and methods for displacing and/or compressing lobes of a prostate gland to relieve pressure on and provide a less obstructed path through a urethra, but there remains an ongoing need in the field of minimally invasive medical devices for improved devices and methods for the manipulation of the prostate gland, as well as tissue in other parts of the anatomy.

The present disclosure addresses these and other needs.

### SUMMARY

Treatment devices, systems, and methods for engaging and manipulating the prostate gland, including the median and lateral lobes, are disclosed herein. The treatment devices include an elongate tissue access assembly attached or coupled to or formed with a handle assembly. The elongate tissue access assembly may be configured to be inserted within an introducer sheath. Embodiments may feature a tissue engagement structure attachable to a distal end portion of the elongate tissue access assembly, along with a loading tool configured to store the tissue engagement structure and facilitate the attachment of the tissue engagement structure to the distal end of the elongate tissue access assembly. The tissue engagement structure may be separate and removable from the elongate tissue access assembly. The tissue engagement structure may thus be configured to attach to, and detach from, the distal end of the elongate tissue access assembly. The attachment/detachment mechanism enables the tissue engagement structure to be coupled and removed from the elongate tissue access assembly when deemed necessary or desired by a clinician, for example before or during a medical procedure. The tissue engagement structure may be configured to transition from a contracted state to an expanded state, for example when a distal portion of the elongate tissue access assembly exits a distal end of the introducer sheath, or in response to one or more actuation mechanisms and/or movement of one or more components or subassemblies of the elongate tissue access assembly.

In some embodiments, a system configured to engage one or more lobes of a prostate gland includes a handle assembly; an elongate shaft assembly attached to the handle assembly; and a tissue engagement structure configured to be attached to and removed from a distal end of the elongate shaft assembly. In some examples, the tissue engagement structure is expandable. In some examples, the tissue engagement structure comprises one or more curved portions or wings. In some examples, the curved portions are configured to flex. In some examples, the system further includes a loading tool configured to house or couple with the tissue engagement structure. In some examples, the loading tool is configured to attach the tissue engagement structure to the distal end of the elongate shaft assembly. In some examples, the loading tool is configured to detach the tissue engagement structure from the distal end of the elongate shaft assembly. In some examples, the loading tool defines an inner cavity configured to receive the tissue engagement structure. In some examples, the loading tool comprises one or more disengagement features configured to facilitate removal of the tissue engagement structure from the elongate shaft assembly.

In some embodiments, a method for compressing prostate tissue involves inserting an introducer sheath longitudinally through a urethra of a subject until at least a distal portion of the introducer sheath is positioned within a prostatic urethra. The method further involves attaching an expandable tissue engagement structure to a distal end of an elongate shaft assembly of a delivery device, and advancing the elongate shaft assembly through the introducer sheath until at least a distal portion of the elongate shaft assembly extends beyond a distal end of the introducer sheath. In some examples, advancing the elongate shaft assembly through the introducer sheath until at least the distal portion of the elongate shaft assembly extends beyond a distal end of the introducer sheath allows the expandable tissue engagement structure to expand. In some examples, the expandable tissue engagement structure comprises curved portions configured to engage and manipulate a prostate gland when expanded outside the introducer sheath. In some examples, the curved portions are biased away from a midline defined by a longitudinal axis of the elongate shaft assembly such that the curved portions are compressed toward the midline when positioned within the introducer sheath and expand away from the midline when positioned outside the introducer sheath. In some examples, the method further involves delivering one or more prostatic implants to the prostate tissue, the one or more prostatic implants configured to compress the prostatic tissue and alleviate constriction of the prostatic urethra. In some examples, the method further involves visualizing at least a portion of the elongate shaft assembly during a procedure using a cystoscope. In some examples, compressing the prostate tissue provides a means for treating benign prostatic hyperplasia.

In some examples, a system configured to engage one or more lobes of a prostate gland includes a handle assembly, an elongate shaft assembly attached to the handle assembly and configured for insertion into a urethra of a subject, and a tissue engagement structure configured to be attached to and removed from a distal end of the elongate shaft assembly. In some examples, the tissue engagement structure is expandable, such that it is configured to transition between an expanded state and a collapsed state. In some examples, the tissue engagement structure comprises one or more curved portions or wings configured to flex. In some examples, the system further includes a loading tool configured to be held by a user and couple with the tissue engagement structure. In some examples, the loading tool is configured to attach the tissue engagement structure to the distal end of the elongate shaft assembly. In some examples, the loading tool is configured to detach the tissue engagement structure from the distal end of the elongate shaft assembly. In some examples, the loading tool defines an inner cavity configured to receive and retain the tissue engagement structure. In some examples, the loading tool comprises one or more disengagement features configured to facilitate removal of the tissue engagement structure from the elongate shaft assembly. In some examples, the loading tool comprises a loading handle assembly. In some examples, the loading handle assembly comprises an inner cavity configured to receive a distal portion of the shaft assembly. In some examples, the loading handle assembly further comprises a removable plug member configured to cover the tissue engagement structure when the tissue engagement structure is positioned within the inner cavity. In some examples, the loading handle assembly further comprises a release mechanism configured to disengage the tissue engagement structure from the elongate shaft assembly upon manual actuation. In some examples, after manual actuation of the release mechanism, the tissue engagement structure cannot be reattached to the elongate shaft assembly. In some examples, the release mechanism comprises a safety lock member and a rotatable lever member. In some examples, the safety lock member comprises a push-button. In some examples, manual depression of the safety lock member enables manual rotation of the rotatable lever member.

In some examples, a loading tool device configured to couple with a tissue engagement structure includes a handheld assembly defining an inner cavity configured to house the tissue engagement structure and receive at least a distal portion of a shaft assembly. The loading tool device may also include one or more manually engageable release members configured to detach the tissue engagement structure from the distal portion of the shaft assembly. In some examples, the handheld assembly is configured to enable attachment of the tissue engagement structure to the distal portion of the shaft assembly. In some examples, the handheld assembly comprises a handle assembly comprising a grip portion and a barrel portion, wherein the inner cavity is within the barrel portion. In some examples, the handle assembly further comprises a release mechanism configured to disengage the tissue engagement structure from the elongate shaft assembly upon manual actuation. In some examples, the release mechanism comprises a pushable safety lock member and a rotatable lever member. In some examples, the inner cavity is further configured to receive at least a portion of a removable plug member, the removable plug member configured to protect and retain the tissue engagement structure within the inner cavity.

In some examples, a method for compressing prostate tissue involves inserting an introducer sheath longitudinally through a urethra of a subject until at least a distal portion of the introducer sheath is positioned within a prostatic urethra, attaching an expandable tissue engagement structure to a distal end of an elongate shaft assembly of an implant delivery device, advancing the elongate shaft assembly through the introducer sheath until at least the distal end of the elongate shaft assembly and the expandable tissue engagement structure attached thereto extend through and beyond a distal opening of the introducer sheath, and delivering one or more prostatic implants through a distal opening of the elongate shaft assembly and into the prostate tissue. In some examples, the expandable tissue engagement structure comprises curved portions configured to transition from a collapsed state to an expanded state when released from the introducer sheath. In some examples, the curved portions are biased away from a midline defined by a longitudinal axis of the elongate shaft assembly such that the curved portions are compressed toward the midline when positioned within the introducer sheath and expand away from the midline when positioned outside the introducer sheath. In some examples, the one or more prostatic implants are configured to compress the prostatic tissue and alleviate constriction of the prostatic urethra. In some examples, attaching the expandable tissue engagement structure to the distal end of the elongate shaft assembly comprises inserting the distal end of the elongate shaft assembly into a cavity defined by a handheld loading tool. In some examples, the expandable tissue engagement structure detaches from the handheld loading tool upon inserting the distal end of the elongate shaft assembly into the cavity. In some examples, the method further involves removing the elongate shaft assembly from the introducer sheath and reinserting the distal end of the elongate shaft assembly into the cavity of the handheld loading tool. In some examples, the method further involves engaging a release mechanism of the handheld loading tool to detach the expandable tissue engagement structure from the distal end of the elongate shaft assembly. In some examples, the release mechanism comprises a manually engageable safety lock member and a manually engageable release member. In some examples, the manually engageable safety lock member comprises a push-button. In some examples, the manually engageable release member comprises a rotatable lever member. In some examples, the manually engageable release member comprises a slidable button. In some examples, the method further involves visualizing at least a portion of the elongate shaft assembly using a cystoscope. In some examples, compressing the prostate tissue provides a means for treating benign prostatic hyperplasia. In some examples, each of the one or more prostatic implants comprises a distal anchor component configured to be inserted through the prostate tissue and deposited against an outer surface of a prostatic capsule, a connector member comprising a suture attached to the distal anchor component and configured to extend through the prostate tissue, and a proximal anchor component attachable to the connector member within the prostatic urethra.

Examples include any method of treating an enlarged prostate disclosed herein. Examples include any method of treating benign prostatic hyperplasia disclosed herein. Examples include any tissue engagement structure, device, or device component disclosed herein. Examples include any method implemented using the tissue engagement structure, device, or device component disclosed herein, along with methods of forming such structures, devices, or device components.

These and other examples and objects of the present devices, systems, and related methods will be set forth in the following Detailed Description. This Summary is intended to provide non-limiting examples of the present subject matter. The Detailed Description below is included to provide further information about the present devices, systems, and related methods. Neither is intended to provide an exclusive or exhaustive explanation of the present devices, systems, and methods because this disclosure is written for those of ordinary skill in the art.

### BRIEF DESCRIPTION OF THE DRAWINGS

In the drawings, like numerals can be used to describe similar features and components throughout the several views. The drawings illustrate generally, by way of example but not by way of limitation, various embodiments discussed in the present patent document.

FIG. 1 is a cross-sectional view of the anatomy surrounding a prostate in a human subject.

FIG. 2 an enlarged cross-sectional view of an area illustrated in FIG. 1, depicting anatomy immediately surrounding and adjacent a prostate.

FIG. 3 is a schematic view of the prostatic anatomy zones.

FIG. 4 is a schematic cross-sectional view of the anatomy zones shown in FIG. 3.

FIG. 5 is a cross-sectional view of a normal prostate.

FIG. 6 is a cross-sectional view of a prostate with enlarged lateral lobes.

FIG. 7 is a cross-sectional view of a prostate with enlarged lateral lobes and an enlarged median lobe.

FIG. 8 illustrates a schematic view of two prostatic implants placed within lateral lobes of a prostate gland in accordance with embodiments disclosed herein.

FIGS. 9A-9G illustrate schematic views of various steps of a method of treating a prostate gland in accordance with embodiments disclosed herein.

FIG. 10A is a side view of an embodiment of a prostatic implant delivery device.

FIG. 10B is an enlarged perspective view of the distal end of the delivery device shown in FIG. 10A.

FIG. 11 illustrates a tissue engagement structure, a loading tool, and a distal end of a shaft assembly in accordance with embodiments disclosed herein.

FIG. 12A illustrates the loading tool and tissue engagement structure shown in FIG. 11.

FIG. 12B illustrates the tissue engagement structure shown in FIGS. 11 and 12A.

FIG. 12C illustrates the tissue engagement structure, loading tool, and shaft assembly shown in FIGS. 11-12B coupled together in accordance with embodiments disclosed herein.

FIG. 12D illustrates the tissue engagement structure attached to a distal end portion of the shaft assembly shown in FIGS. 11-12C.

FIG. 13A illustrates another view of the tissue engagement structure, loading tool, and shaft assembly shown in FIGS. 11-12D.

FIG. 13B illustrates a delivery device coupled with the components shown in FIGS. 11-12D.

FIG. 14A illustrates a loading tool in accordance with embodiments disclosed herein.

FIG. 14B illustrates the loading tool of FIG. 14A coupled with a shaft assembly of a delivery device in accordance with embodiments disclosed herein.

FIG. 15 illustrates a tray-based loading tool in accordance with embodiments disclosed herein.

FIG. 16A illustrates a distal portion of a shaft assembly before and after its attachment to a tissue engagement structure in accordance with embodiments disclosed herein.

FIG. 16B illustrates a close-up view of a distal portion of the shaft assembly and tissue engagement structure shown in FIG. 16A.

FIG. 16C illustrates another close-up view of a distal portion of the shaft assembly and tissue engagement structure shown in FIG. 16A.

FIG. 17 illustrates another embodiment of a tissue engagement structure and associated loading tool in accordance with embodiments disclosed herein.

FIGS. 18A-18C illustrate a process for attaching the tissue engagement structure of FIG. 17 to an elongate shaft assembly in accordance with embodiments disclosed herein.

FIGS. 19A-19C illustrate a process for detaching the tissue engagement structure of FIG. 17 from an elongate shaft assembly in accordance with embodiments disclosed herein.

FIG. 20A illustrates a top plan view of the expandable tissue engagement structure shown in FIG. 17.

FIG. 20B illustrates a perspective view of a distal end portion of a shaft assembly coupled with the expandable tissue engagement structure of FIG. 20A.

FIG. 21 illustrates a perspective view of the interior of a case component of the loading tool shown in FIG. 17.

FIGS. 22A and 22B illustrate two different perspective views of the removable plug member visible in FIG. 17.

FIG. 23 illustrates a perspective view of the safety lock member shown in FIG. 17.

FIG. 24 illustrates a perspective view of a latch member included within the loading tool shown in FIG. 17.

FIGS. 25A and 25B illustrate two different perspective views of the release member of the loading tool shown in FIG. 17.

FIG. 26A illustrates a perspective view of an internal plunger member of the loading tool shown in FIG. 17.

FIG. 26B illustrates a portion of the interior of the loading tool shown in FIG. 17, showing the plunger member of FIG. 26A.

FIG. 27 illustrates another embodiment of a loading tool comprising a loading handle assembly in accordance with embodiments disclosed herein.

FIG. 28 illustrates another embodiment of a tissue engagement structure and associated attachment mechanism in accordance with embodiments disclosed herein.

FIG. 29 illustrates another embodiment of a tissue engagement structure and associated expansion mechanism in accordance with embodiments disclosed herein.

FIG. 30 illustrates another embodiment of a tissue engagement structure and associated expansion mechanism in accordance with embodiments disclosed herein.

FIG. 31 illustrates another embodiment of a tissue engagement structure and associated expansion mechanism in accordance with embodiments disclosed herein.

FIG. 32 illustrates another embodiment of a tissue engagement structure and associated expansion mechanism in accordance with embodiments disclosed herein.

FIG. 33 illustrates another embodiment of a tissue engagement structure and associated expansion mechanism in accordance with embodiments disclosed herein.

FIG. 34 illustrates another embodiment of a tissue engagement structure and associated expansion mechanism in accordance with embodiments disclosed herein.

FIG. 35 illustrates another embodiment of a tissue engagement structure and associated expansion mechanism in accordance with embodiments disclosed herein.

FIG. 36 illustrates another embodiment of a tissue engagement structure and associated expansion mechanism in accordance with embodiments disclosed herein.

FIG. 37 illustrates another embodiment of a tissue engagement structure/mechanism in accordance with embodiments disclosed herein.

FIG. 38 illustrates another embodiment of a tissue engagement structure and associated expansion mechanism in accordance with embodiments disclosed herein.

FIG. 39 illustrates another embodiment of a tissue engagement structure and associated expansion mechanism in accordance with embodiments disclosed herein.

FIG. 40 illustrates another embodiment of a tissue engagement structure and associated expansion mechanism in accordance with embodiments disclosed herein.

FIG. 41 illustrates another embodiment of a tissue engagement structure and associated expansion mechanism in accordance with embodiments disclosed herein.

FIG. 42 illustrates another embodiment of a tissue engagement structure and associated expansion mechanism in accordance with embodiments disclosed herein.

FIG. 43 illustrates another embodiment of a tissue engagement structure and associated expansion mechanism in accordance with embodiments disclosed herein.

FIG. 44 illustrates another embodiment of a tissue engagement structure and associated expansion mechanism in accordance with embodiments disclosed herein.

FIG. 45 illustrates another embodiment of a tissue engagement structure and associated expansion mechanism in accordance with embodiments disclosed herein.

FIG. 46 illustrates another embodiment of a tissue engagement structure and associated expansion mechanism in accordance with embodiments disclosed herein.

FIG. 47 illustrates another embodiment of a tissue engagement structure and associated expansion mechanism in accordance with embodiments disclosed herein.

FIG. 48 illustrates another embodiment of a tissue engagement structure and associated expansion mechanism in accordance with embodiments disclosed herein.

FIG. 49 illustrates another embodiment of a tissue engagement structure and associated expansion mechanism in accordance with embodiments disclosed herein.

FIG. 50 illustrates another embodiment of a tissue engagement structure and associated expansion mechanism in accordance with embodiments disclosed herein.

FIG. 51 illustrates another embodiment of a tissue engagement structure and associated expansion mechanism in accordance with embodiments disclosed herein.

FIG. 52 illustrates another embodiment of a tissue engagement structure and associated expansion mechanism in accordance with embodiments disclosed herein.

FIG. 53 illustrates another embodiment of a tissue engagement structure and associated expansion mechanism in accordance with embodiments disclosed herein.

FIG. 54 illustrates another embodiment of a tissue engagement structure and associated expansion mechanism in accordance with embodiments disclosed herein.

FIG. 55 illustrates another embodiment of a tissue engagement structure and associated expansion mechanism in accordance with embodiments disclosed herein.

FIG. 56 illustrates another embodiment of a tissue engagement structure and associated expansion mechanism in accordance with embodiments disclosed herein.

FIG. 57 illustrates a flow diagram of a method of compressing prostate tissue in accordance with embodiments of the present disclosure.

The drawing figures are not necessarily to scale. Certain features and components may be shown exaggerated in scale or in schematic form and some details may not be shown in the interest of clarity and conciseness.

### DETAILED DESCRIPTION

The present devices, assemblies, systems, and methods provide clinicians with means to treat an enlarged prostate, which may be a symptom of BPH, to alleviate its impingement on the adjacent prostatic urethra. Implants disclosed herein can be placed using a method for compressing a prostate gland or portion thereof. Embodiments include devices and device components configured to anchor at least a portion of an implant delivery device, such as an elongate shaft assembly thereof, at a target location, non-limiting examples of which include a variety of body lumens, just one example of which includes the prostatic urethra.

As used herein, the terms “prostatic implant” and “implant” and “anchor assembly” and “retainer” may be used interchangeably. Each implant, once fully assembled, may include a distal anchor or capsular tab (“CT”), a connector member, portion or suture (“suture”), and a proximal anchor or urethral endpiece (“UE”). The CT and suture may be provided together as a unitary component or assembly, with the CT attached, fixed, integrally formed with, or coupled to one end of the suture. Accordingly, the CT and suture may be referred to as a “CT/suture” or “CT/suture assembly” herein. The UE may be provided as a separate component that is attached to the CT/suture after deploying the CT/suture to the targeted tissue, for instance after the CT has emerged from the distal end of the delivery needle, at or beyond the outer surface of the prostatic capsule, and the suture has been implanted within the prostatic tissue. Embodiments include attaching the UE to the suture of a CT/suture assembly after tightening the CT against the outer capsular surface and tensioning the suture through the targeted tissue. In various examples, the terms “capsular tab” and “CT” and “distal anchor” may be used interchangeably. In various examples, the terms “urethral endpiece” and “UE” and “proximal anchor” may be used interchangeably. In various examples, the terms “connector” and “connector member” and “suture” may be used interchangeably.

The terms “actuator” and “actuator member” may be used interchangeably in some embodiments. In some examples, an “actuator” or “actuator member” may include or be synonymous with a manually engageable portion, member, part, surface, or component (such as a button, lever, trigger, dial, switch, toggle, or knob) configured to be actuated, for example via manually induced movement (such as a lever sweep, button press, or trigger pull).

The term “member” may be used herein to represent a subcomponent or subassembly of a larger component or assembly, or the term may represent the larger component or assembly itself. The terms “element” and “member” and “component” and “portion” may also be used interchangeably herein.

The terms “elongate member” and “tissue access assembly” and “shaft” and “shaft assembly” may refer to the same or similar components and may be used interchangeably herein. Embodiments of a shaft assembly of an implant delivery device may include components configured to access a treatment site within a prostatic urethra, deploy one or more implants, and/or visualize the procedure from within a patient. Examples of the shaft assembly may include a scope tube configured to accommodate an endoscopic instrument within a lumen of the scope tube. Examples of a shaft assembly may include one or more components of a needle assembly, a suture assembly, and/or a cutter assembly, non-limiting examples of which are disclosed in U.S. Pat. No. 11,298,115 and U.S. Patent Application Publication No. 2021/0378658, the entire contents of each of which are incorporated by reference herein.

The term “sheath” may be used interchangeably with “sheath device” and/or “introducer sheath.” Embodiments of the sheaths disclosed herein can be configured to receive an elongate portion, e.g., shaft assembly, of the delivery devices disclosed herein.

The term “procedure” may refer to a medical treatment method used to compress at least a portion of an anatomical structure or tissue, including tissue of the prostate gland, which may be enlarged relative to a corresponding healthy tissue.

The term “user” may refer to a clinician, doctor, nurse, or medical professional performing a procedure described herein, which may involve the implantation of one or more implants within a targeted portion of prostatic tissue, which may be enlarged relative to normal prostatic tissue. In some examples, the term “user” may refer to more than one person, including two or more medical professionals working together to perform a procedure.

The terms “patient” and “subject” may be used interchangeably herein, and may refer to both human and non-human living beings, e.g., animals.

The terms “deploy” and “deliver” and “implant” may be used interchangeably herein, referring to the release and/or ejection of a disclosed implant or subassembly from a delivery device into a tissue being targeted. Accordingly, for instance, an implant may be fully deployed, delivered, or implanted when the CT of an implant is positioned at an outer surface of the prostatic capsule, the suture has been advanced through and tensioned within the targeted prostate lobe, and the UE has been attached to the tensioned suture at the urethral side of the lobe.

Turning now to the figures, which are provided by way of example and not limitation, the present disclosure is directed to devices, assemblies, and systems configured to engage and manipulate tissue within a patient's body. The disclosed devices and components thereof can be employed for various medical purposes including but not limited to retracting, lifting, compressing, approximating, supporting, and/or repositioning tissues, organs, anatomical structures, grafts, or other material found within a patient's body. Such tissue manipulation is intended to facilitate the treatment of diseases or disorders, including, but not limited to, the displacement, compression and/or retraction of prostate tissue, including for instance the lateral lobe(s) and/or median lobe of the prostate gland.

In an aspect of the present disclosure, the tissue engagement or manipulation forms the primary interventional procedure. In other aspects, the tissue engagement or manipulation forms one portion of an interventional procedure, such as the treatment of BPH or for the purpose of retracting, lifting, compressing, approximating, supporting or repositioning other anatomy or for the purpose of retracting, lifting, compressing, approximating, supporting, or repositioning a first section of anatomy with respect to a second section of anatomy. In some aspects, tissue engagement secures or improves the desired positioning of a tissue access assembly for precise, targeted delivery of one or more implants to prostate tissue.

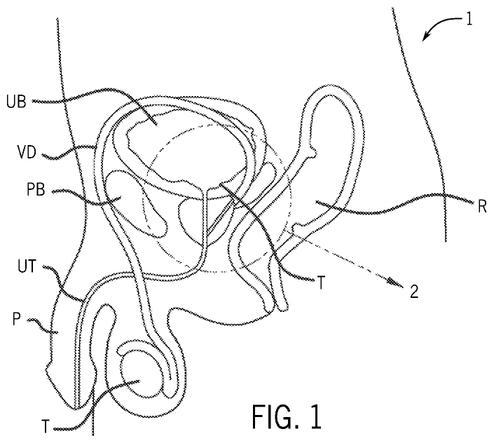

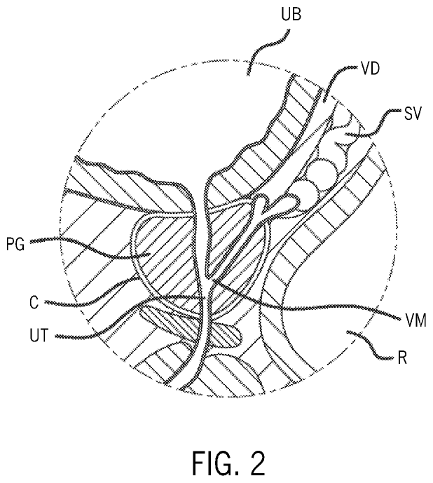

With reference to FIGS. 1-4, various features of urological anatomy of a human male subject 1 are presented, with FIG. 2 providing a magnified view of detail 2. The prostate gland PG is a walnut-sized gland located adjacent the urinary bladder UB. The urethra UT runs through the prostate gland PG and the penis P. The prostate gland PG secretes fluid that protects and nourishes sperm. The prostate gland PG also contracts during ejaculation to expel semen and to provide a valve to keep urine out of the semen. A capsule C surrounds the prostate gland PG.

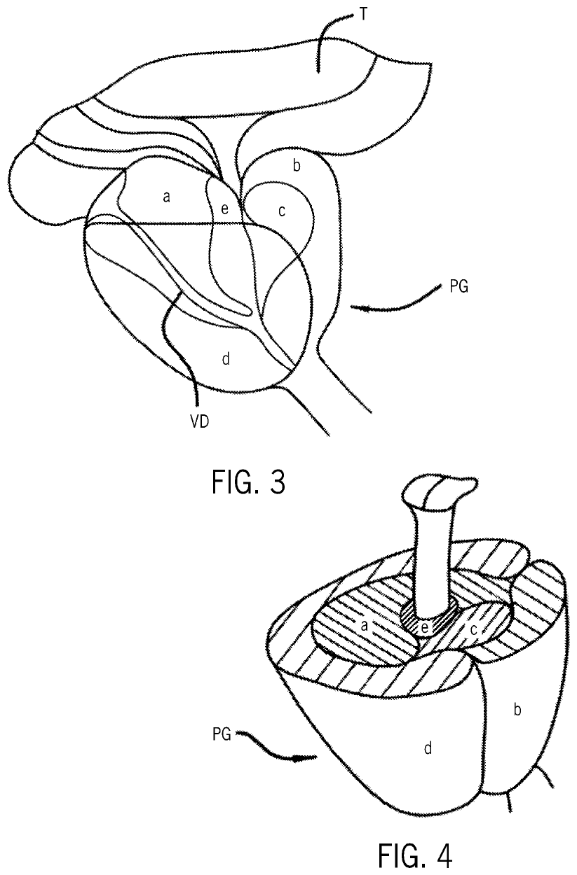

The urinary bladder UB holds urine. The vas deferentia VD define ducts through which semen is carried, and the seminal vesicles SV secrete seminal fluid. The rectum R is the end segment of the large intestine through which waste is dispelled. The urethra UT carries both urine and semen out of the body. Thus, the urethra is connected to the urinary bladder UB and provides a passageway to the vas deferentia VD and seminal vesicles SV. The verumontanum VM is a crest in the wall of the urethra UT where the seminal ducts enter. The prostatic urethra is the section of the urethra UT which extends through the prostate. The trigone T (see FIG. 3) is a smooth triangular region of the bladder. It is sensitive to expansion and signals the brain when the urinary bladder UB is full.

The prostate gland can be classified by zones or described by referring to its lobes (see FIG. 4). Whereas the zone classification is typically used in pathology, the lobe classification is more often used in anatomy. The central zone (a) of a prostate gland PG is about 25% of a normal prostate and this zone surrounds the ejaculating ducts. There is some prevalence of BPH in the transition zone. The fibromuscular zone (b) is usually devoid of glandular components and, as its name suggests, is composed of muscle tissue and fibrous tissue. The transitional zone (c) generally overlays the proximal urethra and is the region of the gland that grows throughout life. This zone is often associated with the condition of benign prostatic enlargement. Finally, the peripheral zone (d) is the sub-capsular portion of the posterior aspect of the prostate gland that surrounds the distal urethra.

The lobe characterization is different from the zone characterization, but there is some overlap. The anterior lobe is devoid of glandular tissue and is formed of fibromuscular tissue. The anterior lobe roughly corresponds to the anterior portion of the transitional zone (c). The posterior lobe roughly corresponds to the peripheral zone (d) and can be palpated through the rectum during a digital rectal exam. The posterior lobe is the site of 70-80% of prostatic cancers. The lateral lobes are the main mass of the prostate and are separated by the urethra. All pathological zones may be present in the lateral lobes. Lastly, the median lobe roughly corresponds to part of the central zone. It varies greatly in size from subject to subject and in some cases is devoid of glandular tissue.

A large or enlarged median lobe can act as a ball valve, blocking the bladder neck, or opening, into the urethra. Various approaches are contemplated to address such a condition. It is contemplated that the median lobe can be compressed, displaced and/or retracted to eliminate or decrease the blocking of the bladder neck opening.

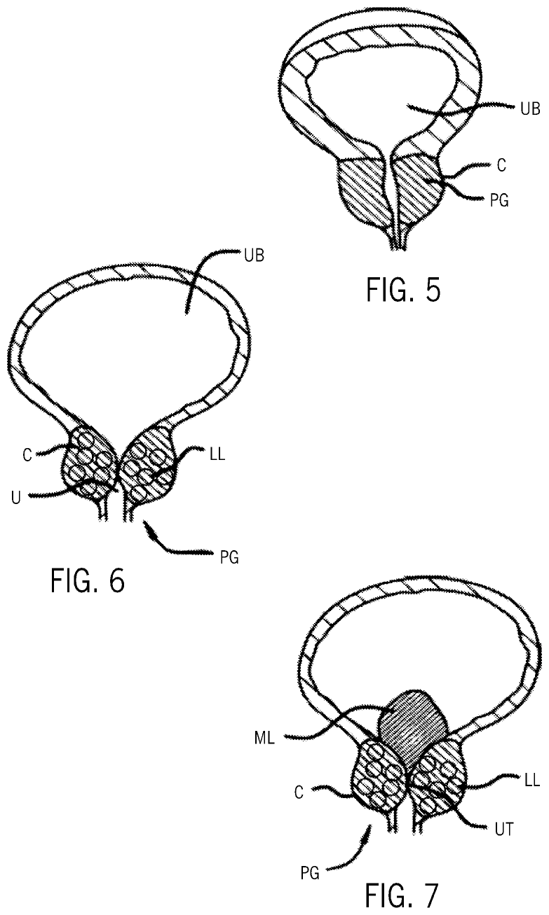

Turning now to FIGS. 5-7, there are shown various prostate glands in cross-section. FIG. 5 depicts the urinary bladder UB and prostate gland PG of a healthy subject. FIG. 6 illustrates an individual with a prostate having enlarged lateral lobes LL, and FIG. 7 depicts a subject suffering from both enlarged lateral lobes LL and an enlarged median lobe ML. It is to be appreciated that such enlarged anatomy impinges on the urethra UT and affects normal bladder functioning. The following devices and approaches can be employed to access and manipulate prostatic tissue during an interventional or diagnostic procedure.

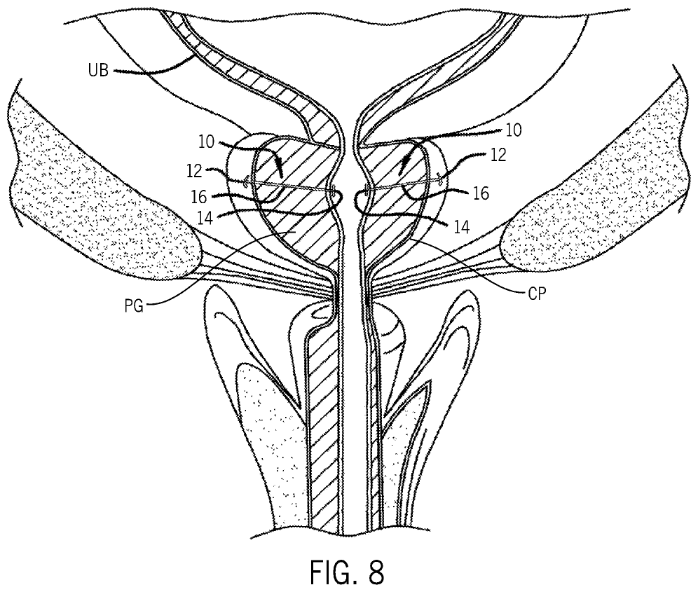

By way of example, FIG. 8 illustrates a coronal section through the lower abdomen of a male human suffering from BPH, showing a hypertrophied prostate gland treated with embodiments of the device and device components disclosed herein. The enlarged prostate gland is compressible and can be retracted so as to relieve the pressure from the urethra. In accordance with one embodiment of the present invention, a retaining device, retainer, or implant (hereinafter “implant”) can be placed through the prostate gland in order to relieve the pressure on the urethra. FIG. 8 shows two implants 10 previously implanted in the prostate gland. Each implant 10 includes a distal anchor or capsular tab 12 (or “CT”) and a proximal anchor or urethra endpiece (or “UE”) 14. After assembly of the implant 10, the CT 12 and UE 14 are connected by a middle portion or connector 16, which may comprise, include, or resemble a suture. The radial distance from the urethra to each CT 12 is greater than the radial distance from the urethra to each UE 14. The distance or tension between the anchors is sufficient to compress, displace or change the orientation of an anatomical region between the CT 12 and UE 14. The connector 16 may be substantially inelastic so as to maintain a constant force or distance between the UE and CT, or it may be elastic to facilitate drawing the UE and CT closer together. In the embodiment shown in FIG. 8, each CT 12 is located on the outer surface of the capsule CP of the prostate gland and acts as a capsular anchor. Alternatively, the CT 12 may be embedded inside the tissue of prostate gland PG, or in the surrounding structures around the prostate, such as periosteum of the pelvic bones, within the bones themselves, pelvic fascia, coopers ligament, muscles traversing the pelvis or bladder wall. Also, in the embodiment shown in FIG. 8, the UE 14 may be located on the inner wall of urethra UT, where it acts as a urethral anchor. Alternatively, the UE 14 may be embedded inside the tissue of prostate gland PG or surrounding structures as outlined above. The CT 12 and UE 14 are implanted in the anatomy such that a desired distance or tension is created in connector 16. This causes the CT 12 and UE 14 to retract or compress a region of prostate gland PG to relieve the urethral constriction. In FIG. 8, two implants 10 are implanted in a lateral lobe (side lobe) of prostate gland PG. The various methods and devices disclosed herein may be used to treat a single lobe or multiple lobes, including one or more lateral lobes and the median lobe, of the prostate gland or other anatomical structures, by deploying one or more implants to the targeted tissue. Non-limiting examples of each implant 10 can also be found in U.S. Pat. No. 11,298,115, which is incorporated by reference herein, including the embodiment shown in FIG. 3 thereof, among others disclosed therein and described in this disclosure.

The implants 10 may be deployed at particular angles relative to the axis of the urethra to target one or more lateral lobes and/or median lobe of the prostate gland. For example, implant 10 may be deployed between the 1 o'clock and 3 o'clock position relative to the axis of the urethra to target the left lateral lobe of the prostate gland. In another example, implant 10 may be deployed between the 9 o'clock and 11 o'clock position relative to the axis of the urethra to target the right lateral lobe of the prostate gland. In another example, implant 10 may be deployed between the 4 o'clock and 8 o'clock position relative to the axis of the urethra to target the middle lobe of the prostate gland.

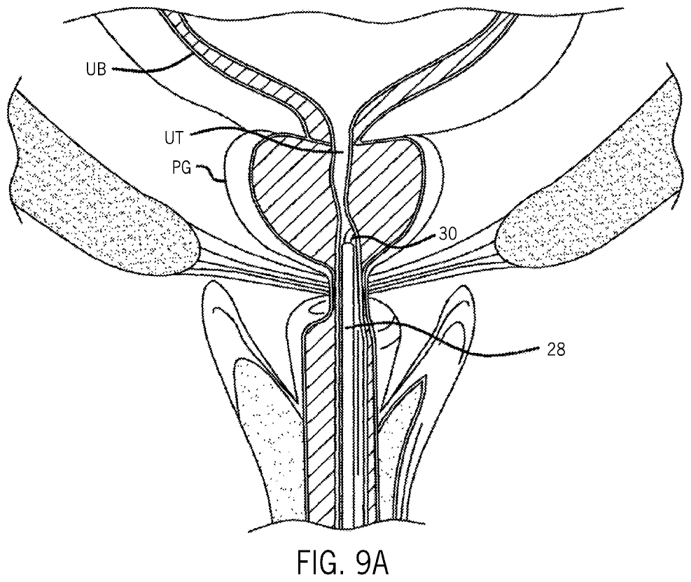

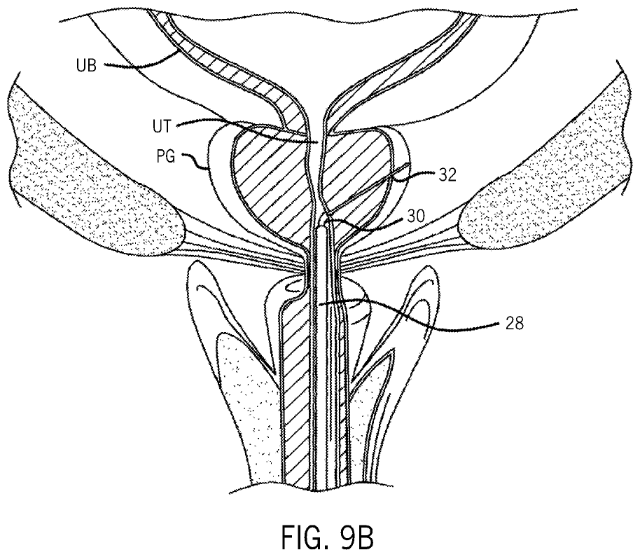

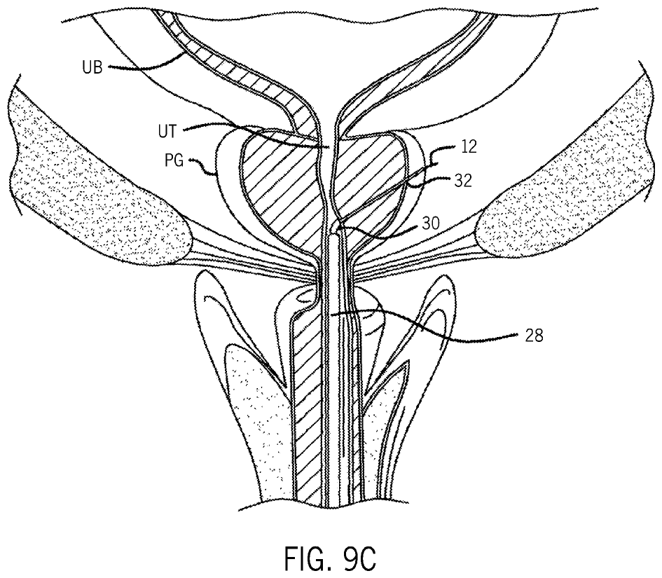

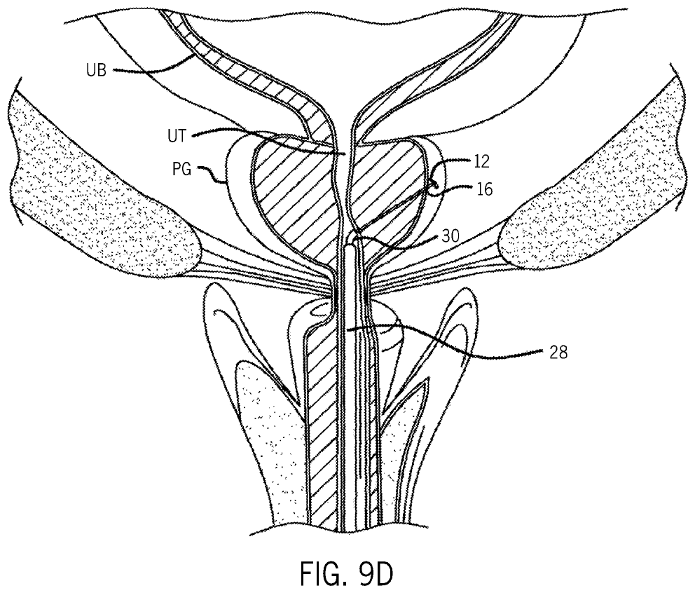

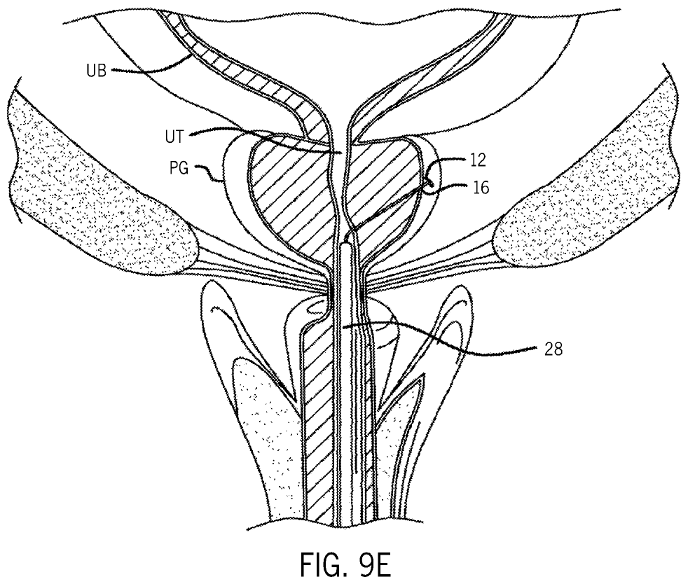

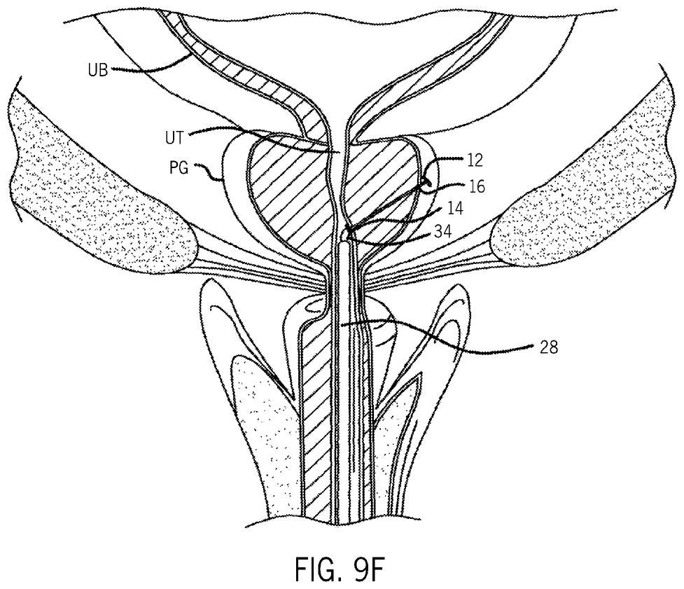

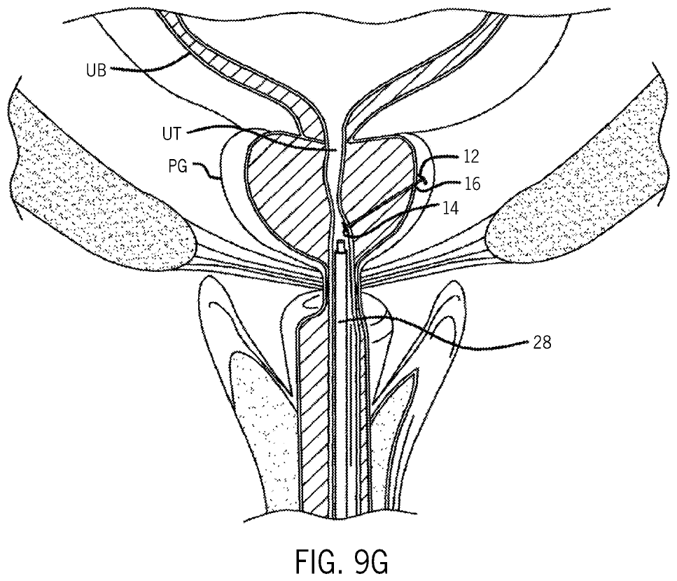

FIGS. 9A-9G show the various steps of a non-limiting example of a method of treating a prostate gland, which may be enlarged, using one or more of the implants and associated implant delivery devices disclosed herein. One or more of the illustrated steps may be modified or excluded in accordance with certain embodiments disclosed herein, for example those not requiring a sheath. One or more of the depicted steps, shown for illustrative purposes, may be performed in a different manner depending on the particular delivery device(s) used, embodiments of which are disclosed herein. Similar methods may also be implemented to deploy retainers, implants, or compression devices in other anatomical structures.

In the step shown in FIG. 9A, an elongate, tubular sheath 28, which may comprise a standard resectoscope sheath, may be introduced into the urethra (trans-urethrally). The sheath 28 may be advanced through the urethra UT until the distal end of the sheath 28 is positioned within or near a region of the urethra UT that is obstructed by a hypertrophied prostate gland PG (prostatic urethra). A distal anchor delivery device or elongate delivery shaft assembly 30 may then be introduced and advanced through the inner lumen of the sheath 28. The shaft assembly 30 may be placed in the sheath 28 after the distal end of sheath 28 is positioned near or within the region of the urethra UT that is obstructed, or the shaft assembly 30 may be pre-loaded in the sheath 28 before positioning of the sheath 28. The shaft assembly 30 may be advanced through the sheath 28 until the distal end of the shaft assembly 30 emerges out of the distal end of the sheath 28. The shaft assembly 30 may be oriented such that a working channel opening of the shaft assembly 30 points toward a lateral lobe of prostate gland PG.

In the step shown in FIG. 9B, a penetrating member or hollow needle 32 may be introduced and/or extended through the shaft assembly 30. The needle 32 may be placed in the shaft assembly 30 after the shaft assembly 30 is advanced through sheath 28, or the needle 32 can be pre-loaded in the shaft assembly 30. In one non-limiting embodiment, the needle 32 is a 20-gauge needle, although other sizes may be used. The needle 32 is advanced through the shaft assembly 30 such that it emerges through the working channel opening. The needle 32 may be further advanced until it penetrates through the tissue of the prostate gland PG and the distal end of needle 32 emerges out of the prostatic capsule CP of the prostate gland PG.

In the step shown in FIG. 9C, the CT 12 connected to the connector 16 may be advanced through the needle 32 and deployed at, or just beyond, the outer surface of the prostatic capsule. In some embodiments, the CT 12 may be deployed by being extended beyond the distal end of the needle 32. In other embodiments, the CT 12 may be deployed by being held in place by a pusher or connector while the needle 32 is retracted, thus exposing the CT and unsheathing the connector 16 upon continued needle retraction. The CT 12 can be pre-loaded within the needle 32 or it can be loaded in the needle 32 after the needle 32 has been advanced through shaft assembly 30.

In the step shown in FIG. 9D, the needle 32 may be removed from the prostate gland PG and back into the shaft assembly 30 by pulling the needle 32 in the proximal direction. In the step shown in FIG. 9E, the shaft assembly 30 may be removed from the sheath 28 by pulling the shaft assembly 30 in the proximal direction. Also, the connector 16 may be pulled to orient the CT 12 approximately perpendicularly or otherwise transverse to the connector 16, against the outer surface of the prostatic capsule. In some embodiments, the shaft assembly 30 may remain positioned in the manner shown in FIG. 9D until the implantation process is complete, e.g., after UE 14 attachment to the connector 16.

In the step shown in FIG. 9F, the connector 16 may be passed through the UE 14 located on a proximal anchor delivery device 34, which in some examples may constitute one or more components or portions of the shaft assembly 30. In some embodiments, the proximal anchor delivery device 34 may be advanced through the sheath 28 such that the distal end of proximal anchor delivery device 34 emerges out of the distal end of sheath 28. A desired tension may be introduced in connector 16 such that CT 12 is pulled proximally by the connector 16 with a desired force. The UE 14 can additionally or alternatively be visualized through an endoscope or under fluoroscopy and advanced along the connector 16 until the desired retraction of the tissue is achieved. In other embodiments, the UE 14 may be a v-shaped or clothespin-shaped piece that may be forced, in some cases at high speed, onto the connector 16 to fixedly engage the connector.

In the step shown in FIG. 9G, the UE 14 is attached to the connector 16. The UE 14 is also released from the proximal anchor delivery device 34, thus deploying the UE 14 and the implant 10 as a whole in the targeted anatomy. The proximal anchor delivery device 34, shaft assembly 30, and sheath 28 may then be removed from the anatomy, retracting proximally through the urethra.

This method may be used to retract, lift, support, reposition or compress multiple regions or lobes of the prostate gland PG. In the method shown in FIGS. 9A-9G, the CT 12 is deployed on the outer surface of the prostatic capsule. Thus, the CT 12 acts as a capsular anchor. Alternatively, the CT 12 may be deployed inside the tissue of prostate gland PG or beyond the prostate as outlined previously. Similarly, in the method shown in FIGS. 9A-9G, the UE 14 may be deployed on the inner wall of urethra UT, acting as a urethral anchor. Alternatively, the UE 14 may be deployed inside the tissue of prostate gland PG. In some examples, as noted above, one or more of the delivery components or devices may not be used or necessary, non-limiting examples of which may include sheath 28. During the illustrated method, attaining and maintaining the proper positioning of the shaft assembly 30, particularly its distal end, may be critical to the successful execution of the method. The tissue engagement structures and associated loading tools disclosed herein are configured to enhance anchoring of the shaft assembly 30 at the target site during a procedure, thereby positioning the shaft assembly 30 for effective deployment of each implant 10.

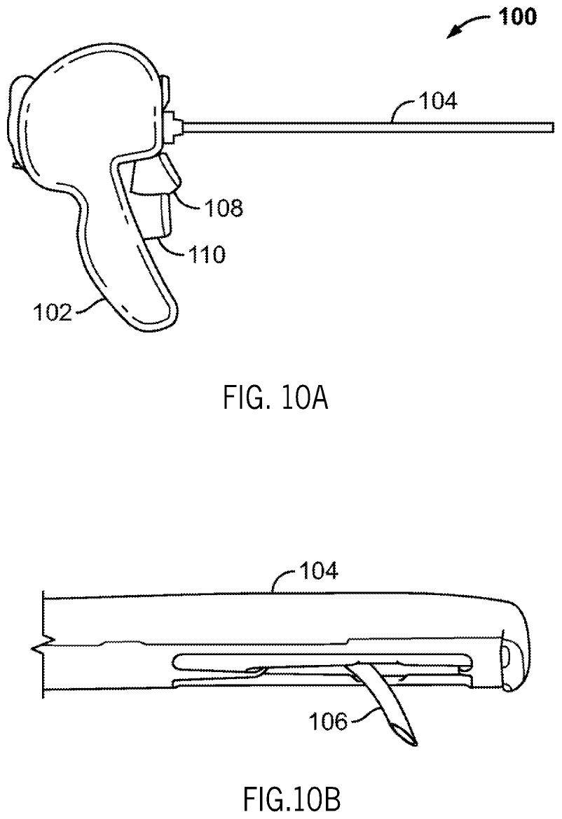

Referring now to FIGS. 10A and 10B, a non-limiting example of an implant delivery or treatment device 100 is shown. The treatment device 100 can include a handle assembly 102 and an elongate tissue access assembly or delivery shaft assembly 104 (hereinafter “shaft assembly 104”), the distal end of which is magnified in FIG. 10B. Like the shaft assembly 30 depicted in FIGS. 9A-9D, the shaft assembly 104 is a low-profile component configured to access an interventional site by being advanced longitudinally through the urethra, where the shaft assembly 104 may also engage and manipulate target tissue. One or more components of a prostatic implant, e.g., CT, suture, and/or UE, may be advanced through and/or positioned within the shaft assembly 104. The treatment device 100 can be configured to assemble and implant one or more anchor assemblies or implants (e.g., implant(s) 10) within a patient's body, for example within and against prostate tissue. The treatment device 100 is further contemplated to be compatible for use with minimally invasive techniques (such as cystoscopy), such that a patient can tolerate a procedure while awake or under light sedation rather than under general anesthesia. The treatment device 100 additionally includes structures configured to receive a conventional remote viewing device (e.g., an endoscope) so that the steps being performed at the interventional site can be observed by the physician.

The shaft assembly 104 can house members, components, and subassemblies to manipulate target tissue, such structures including but not limited to a needle assembly 106 defining a lumen and configured to pierce through tissue (see also needle 32). The shaft assembly 104 can also be equipped with features to manipulate target tissue and/or stabilize the delivery device 100 at its interventional site, for example within a patient's urethra adjacent to one or more lobes of a prostate gland (also referred to as the “prostatic urethra” herein). The shaft assembly 104 can be inserted through a sheath of a size compatible with conventional cystoscopy, including sizes such as 19F or 21F, among others. The shaft assembly 104 can be rigid or flexible. In some embodiments, the shaft assembly 104 is sufficiently rigid (or can be made sufficiently rigid when at the interventional site) to allow manual compression of tissue at the interventional site by leveraging or pushing the handle assembly 102. Various embodiments of the treatment device 100 can include subassemblies and components to dissect, resect, or otherwise alter a prostatic lobe, including one or more components for cutting a suture and attaching a urethral endpiece thereto (e.g., UE 14).

The treatment device 100 may include one or more manually engageable actuators configured to deploy the implants in response to user engagement. Examples of such actuators may include one or more trigger members, non-limiting examples of which are shown in FIG. 10A in the form of first trigger 108 and second trigger 110. Upon manual actuation, the first trigger 108 and second trigger 110 may drive discrete steps of the implant deployment process, such as extension of the needle assembly 106 though a prostate gland, attachment of a urethral endpiece to the suture, and/or cutting of the surplus suture within the urethra after attachment of the urethral endpiece.

In one particular, non-limiting use in treating a prostate, the shaft assembly 104 of the delivery device 100 may be placed within a urethra leading to a urinary bladder of a patient. In one approach, the shaft assembly 104 can be placed within an introducer sheath previously positioned in the urethra (e.g., sheath 28), or alternatively, the shaft assembly 104 can be inserted directly within the urethra. When employing an introducer sheath, the sheath can be attached to a sheath mount assembly. The sheath is advanced within the patient until a leading end of the sheath reaches the prostatic urethra. In a specific approach, a first side (i.e., lateral lobe) of the prostate to be treated is chosen while the shaft assembly 104 extends through the bladder and is turned accordingly. The distal end of the shaft assembly 104 can be used to depress the urethra into the prostate gland by compressing the inner prostate tissue. The inside of the prostate gland (i.e., adenoma) is spongy and compressible, while the outer surface (i.e., capsule) of the prostate gland (opposite the urethra) is firm. By pivoting the shaft assembly 104 laterally about the pubic symphysis PS relative to the patient's midline, the physician can depress the urethra into the prostate gland, thereby compressing the adenoma and creating the desired opening through the urethra. The needle assembly 106, led by its distal tip, may be inserted into and through the adjacent prostate tissue with one or more implant assemblies or components thereof housed within the lumen defined by the needle assembly 106. The implants may be configured to compress the prostate tissue after their delivery, thereby relieving constriction of the prostatic urethra caused by enlargement of the tissue. Further details and background concerning related and complementary interventional procedures are described in various U.S. patents, including but not limited to U.S. Pat. Nos. 8,491,606 and 8,758,366 and 12,121,228, the entire of the contents of which are hereby incorporated by reference.

In various embodiments, each implant or component thereof may be provided in a separate delivery device or cartridge configured to couple therewith. Where a cartridge containing an implant is used, the cartridge may be loaded into a delivery device, which may then be activated by a user to deploy the implant into the targeted tissue by transferring mechanical energy to the cartridge or internal subassembly. The delivery device, or at least its elongate delivery shaft assembly, may then be then removed from the patient, and the spent cartridge replaced with a new cartridge containing a second implant. The delivery device (or more specifically, the shaft assembly) may then be re-inserted and the deployment process repeated. Multiple implants are often necessary to complete a single procedure, thus necessitating multiple cartridge exchanges and the associated removals/reinsertions of the shaft assembly. In some embodiments, a delivery device may be configured to simultaneously hold and serially deploy multiple implants or be reloaded with multiple implants during a procedure, for example via cartridge exchange, in a manner that does not require the shaft assembly to be removed between deployments. Embodiments of the delivery devices disclosed herein may thus be configured to deploy multiple implants without removing the elongate shaft assembly from the urethra of a patient. According to such embodiments, which may be considered multi-fire embodiments, the delivery devices include various subassemblies mobilized via one or more actuators or manually accessible structures, the operation of which is coordinated and synchronized to ensure accurate and precise implantation of each implant. Non-limiting examples of such multi-fire devices and device components are described in U.S. Patent Application Publication No. 2025/0339134, the entire contents of which are incorporated by reference herein.

Embodiments of one or more implant delivery systems disclosed herein may include one or more visualization components, assemblies, or systems configured to enable real-time viewing of a procedure at or near a treatment site, for instance within or near a prostatic urethra. In some examples, one or more visualization components can be included, integrated, or otherwise coupled with one or more components of the implant delivery device, such as within at least a portion of the elongate shaft assembly, where the component(s) can provide a real-time view of the procedure from within the patient. Examples may provide a view of the needle exiting the device and advancing into the targeted prostatic lobe. Examples may also be configured to provide a real-time view of the suture tensioning and/or UE attachment process. Non-limiting examples of various visualization components, assemblies, and systems are described in U.S. Patent Application Publication No. 2025/0339139, the entire contents of which are incorporated by reference herein.

When a treatment device is used at an interventional site, such as within the prostatic urethra, manipulating the treatment device into a position conducive to receiving an implant prior to deployment may be critical. Precise positioning of the distal end of the shaft assembly (e.g., shaft assembly 30), in particular, may be necessary for effective implant placement, and maintaining the initial position may be difficult for a variety of reasons, examples of which may include slippage of the shaft assembly within the anatomy caused by patient movement and/or clinician movement during a procedure. Embodiments of a device or device accessory and associated method of use that can position and stabilize a treatment device to better engage and manipulate target tissue, including the lateral and/or median lobes of the prostate, are described below. In some embodiments, such a device can include a winged, expandable/collapsible structure that increases the distal surface area of the shaft assembly to engage and manipulate tissue, thereby anchoring the device at the desired position. The increased footprint of tissue engagement enables improved control when treating challenging anatomies, such as obstructive median lobes or large lateral lobes, and in patients experiencing tissue regrowth after transurethral resection of the prostate (TURP). The tissue engagement structure may be reversibly coupled to the distal end of the delivery shaft assembly in some embodiments, such that the tissue engagement structure may be attached and/or removed before or during a treatment procedure, for example in response to a clinician's determination that increased tissue engagement within the prostatic urethra is necessary or desired. The ability to attach and/or detach the tissue engagement structure provides improved, real-time flexibility for clinicians performing a procedure.

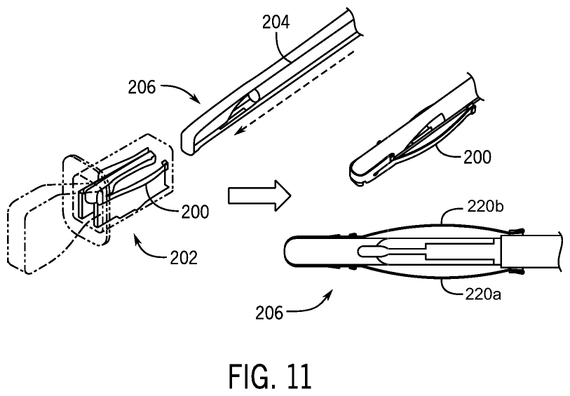

FIG. 11 illustrates a tissue engagement structure, which may be configured to expand (hereinafter “expandable member”), that is coupled, stored, pre-installed, and/or otherwise positioned within an assembly tool or handheld loading tool (hereinafter “loading tool”). In particular, expandable member 200 is shown within loading tool 202. The loading tool 202 facilitates attachment and detachment of the expandable member 200 to and from an elongate delivery shaft assembly 204 (hereinafter “shaft assembly”) of a delivery device (e.g., delivery device 100) at a distal end 206 of the shaft assembly 204. Unlike preexisting devices, the expandable member 200 in the embodiment shown in FIG. 11 is not welded or fixed to the shaft assembly 204. It is instead a separate component configured to be attached to, and removed from, the distal end 206 of the shaft assembly 204. Engagement of the loading tool 202 with the shaft assembly 204 enables clinicians to attach and detach the expandable member 200 as desired without switching delivery devices during a procedure. For instance, if a clinician determines that increased or enhanced engagement between prostate tissue and the distal end 206 of the shaft assembly 204 is necessary or desired, the shaft assembly 204 can be removed from the patient, the expandable member 200 attached to the distal end 206 of the shaft assembly 204, and the same shaft assembly then reinserted and advanced distally through the urethra, e.g., through an introducer sheath. When the shaft assembly 204 is inserted into the sheath, the expandable member 200 may reduce in profile, for example via collapsing of the curved portions or “wings” toward the midline of the longitudinal axis of the treatment device. The expandable member 200 can then be expanded at the desired position within the urethra, for example after the distal end portion 206 of the shaft assembly 204 now attached to the expandable member 200 is extended distally beyond the opening at the distal end of the surrounding introducer sheath. Once expanded, the expandable member 200 may anchor the shaft assembly 204 within the prostatic urethra in atraumatic fashion. Advancement of the expandable member 200 through and beyond the distal end of the delivery sheath may be driven via manual actuation of the handle assembly of the delivery device or member thereof, e.g., a trigger (see e.g., FIG. 10A). As the expandable member 200 exits the sheath, the expandable portions or wings expand or spring open, contacting the targeted tissue without piercing, cutting, or otherwise damaging the same. The expandable member 200 can thus be a separate component or accessory to the delivery device. The delivery system or components thereof can be sold separately or packaged together, e.g., in a kit. The expandable member 200 may be pre-loaded or stored within the loading tool 202 in some embodiments.

To attach the expandable member 200 to the shaft assembly 204, the loading tool 202 (with the expandable member 200 in tow) can be urged onto, slid over, or otherwise engaged with the distal end 206 of the shaft assembly 204, at which point the expandable member 200 may attach to the shaft assembly 204 and disengage from the loading tool 202. The empty loading tool 202 can be uncoupled from the shaft assembly 204, leaving the expandable member 200 attached to the distal end 206 of the shaft assembly 204.

The expandable member 200 may be subsequently removed from the distal end 206 of the shaft assembly 204 by reinserting the distal end 206 of the delivery shaft assembly 204 into the loading tool 202. Detachment of the expandable member 200 from the shaft assembly 204 may involve pinching or squeezing disengagement features present on the loading tool 202, e.g., in a manner analogous to inserting a pen into its complementary cap (and subsequently detaching the two components). The curved elongate portions or “wings” of the attached expandable member 200 in its deployed state may protrude laterally outward, away from the longitudinal axis of the shaft assembly 204. To detach the expandable member 200 from the shaft assembly 204, the loading tool 202 may be re-engaged with the expandable member 200, for example via sliding the receiving cavity of the loading tool 202 over the expandable member 200 and distal portion 206 of the shaft assembly 204 and locking the two components together.

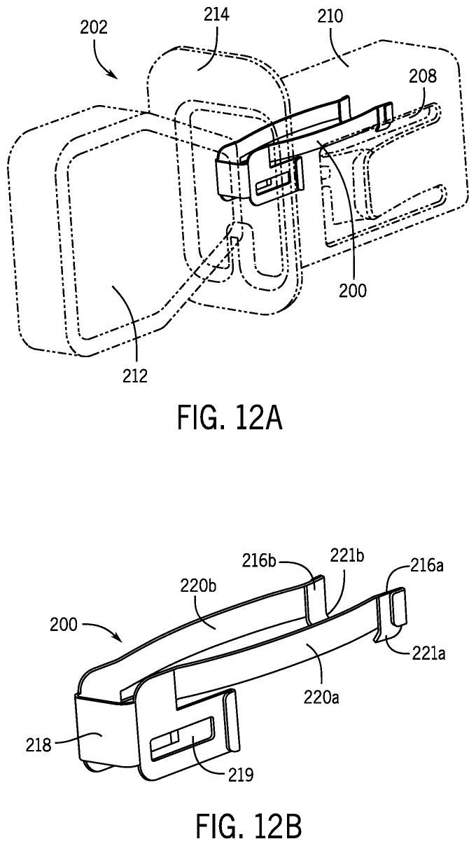

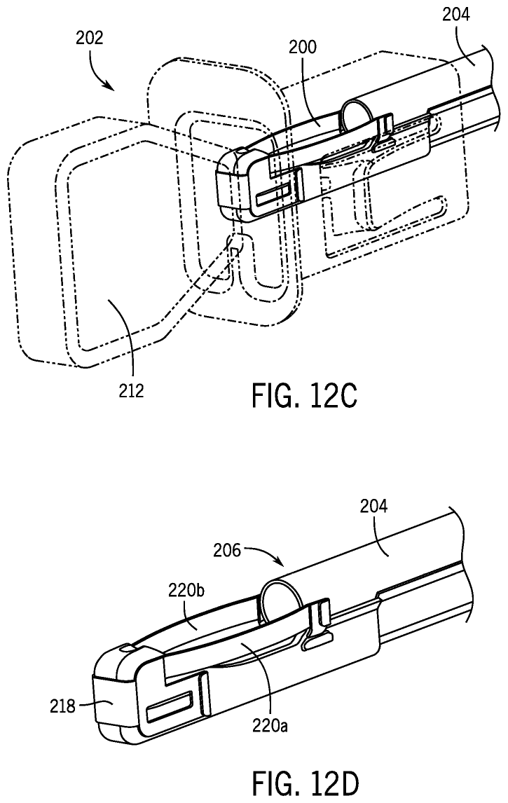

FIGS. 12A-12D provide additional views of the expandable member 200 and complementary loading tool 202. In FIG. 12A, the loading tool 202 is shown in transparency to reveal its inner structure in accordance with various embodiments. The expandable member 200 is positioned within the loading tool 202, stored within a receiving or holding cavity 208 defined by a coupling portion 210 of the loading tool 202. The loading tool 202 also includes a manually engageable portion, here in the form of a tab 212 sized and configured to be grasped or pinched by the fingers of a clinician holding or otherwise manipulating the loading tool 202, for example during attachment/detachment of the expandable member 200. A wall portion or partition 214 is defined between the tab 212 and the coupling portion 210 for improved handling.

FIG. 12B depicts the expandable member 200 in isolation. The expandable member 200 may include expandable and non-expandable portions in some embodiments. The terms “expandable member” and “tissue engagement structure” may be used interchangeably. In some examples, the expandable member may constitute a portion or component of a tissue engagement structure. The expandable member 200 may include two arms or proximal end segments 216a, 216b situated in parallel (or substantially in parallel) and connected by a distal connecting portion 218 or component. Connected or formed with the proximal end segments 216a, 216b are two curved, expandable portions 220a, 220b. The expandable curved portions 220, 220b (which may also be referred to as “wings”) can be configured to flex and may be biased toward an expanded/deployed configuration, depicted in FIG. 12B, in which each expandable portion extends or protrudes outwardly, away from the longitudinal axis of the expandable member 200 and shaft assembly 204 couplable thereto. The expandable member 200 may include or define one or more windows or cutouts 219 configured to receive a protruding feature of the shaft assembly 204, thereby securing the reversible engagement of the expandable member 200 with the shaft assembly 204. The proximal end of the expandable member 200 may include tabs 221a, 221b, which may also be configured to engage with complementary features of the shaft assembly 204, further securing their engagement. Embodiments of the expandable member 200 may include features similar to the expandable members disclosed in U.S. Pat. No. 12,121,228 (e.g., expandable member 200 disclosed therein), the entire contents of which are incorporated by reference herein.

Because embodiments of the expandable member 200 may not be welded, the expandable member 200 (or curved portions 220a, 220b) may be manufactured from stainless steel or nitinol in some examples. The expandable member 200 (and other expandable members disclosed herein) can be reused during a procedure, such that device exchanges are not required between expandable member deployments. The attachment/detachment capabilities of the expandable members disclosed herein also separate the cost of the expandable member from the delivery system as a whole (e.g., system 100). This may be especially advantageous in embodiments featuring a delivery device configured to simultaneously hold and serially deploy multiple implants or be reloaded with multiple implants during a single procedure, for example via cartridge exchange, in a manner that does not require the shaft assembly to be removed between deployments.

FIG. 12C illustrates the loading tool 202 coupled to distal end 206 of the shaft assembly 204, such that the distal end 206 of the shaft assembly 204 has been inserted into the holding cavity 208 of the loading tool 202, where the expandable member 200 is positioned. Facilitated by manual engagement with the loading tool 202, for example via grasping or gripping the tab 212, the distal end 206 of the shaft assembly 204 and the proximal end segments 216a, 216b of the expandable member 200 may be attached to each other, for example via snap fit or friction fit. After or during attachment of the expandable member 200 to the shaft assembly 204, the expandable member 200 may be released from the loading tool 202. The loading tool 202 can be disengaged or otherwise removed from the distal end 206 of the shaft assembly 204, for example via sliding or pulling the loading tool 202 and delivery shaft 204 apart. The expandable member 200 remains attached to the distal end of the shaft assembly 204, as shown in FIG. 12D. In some embodiments, the empty loading tool 202 may be reloaded with another expandable member 200. In some embodiments, the loading tool 202 may be used again for detachment of the expandable member 200 from the shaft assembly 204, for example if/when a clinician determines that the expandable member 200 is not necessary or desirable for a part of a treatment procedure, or after the procedure has been completed. The ability to attach and remove expandable members as desired during a procedure may increase user flexibility, improve patient comfort, reduce average procedure times, and minimize waste.

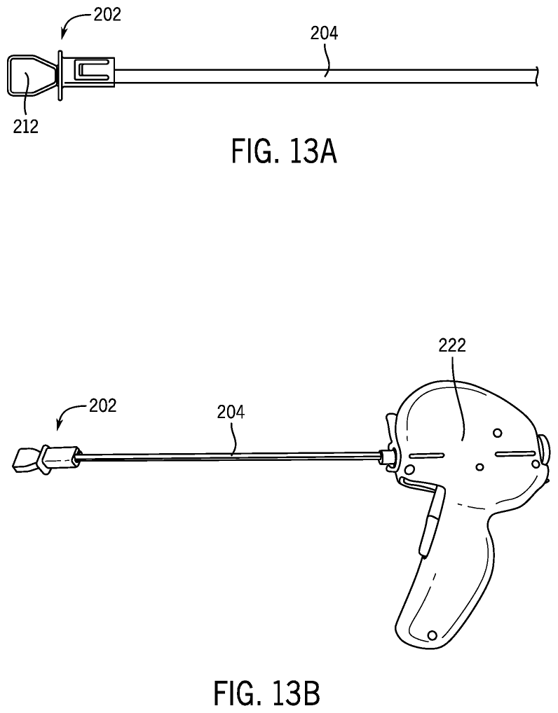

For additional scale, FIGS. 13A and 13B show the loading tool 202 coupled with the distal end of the shaft assembly 204, which is attached at its proximal end to the manually engageable handle assembly 222 of a delivery device.

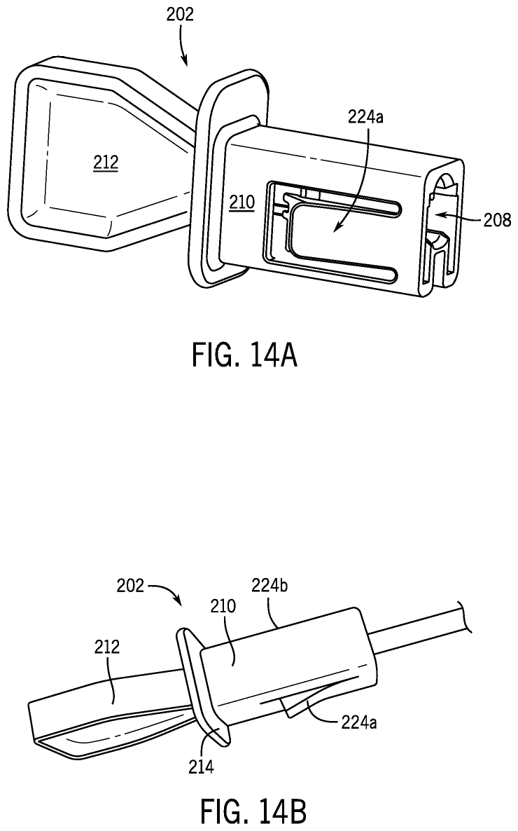

The loading tool 202 may include one or more disengagement features configured to facilitate detachment of the expandable member 200 from the shaft assembly 204. FIG. 14A illustrates one example in the form of protrusions or tabs 224a, 224b (only 224a is visible in the depicted orientation) configured to allow or facilitate removal of the expandable member 200 from the shaft assembly 204 in response to manual engagement with the tabs 224a, 224b. The tabs 224a, 224b may be configured to flex and may be biased toward an outward configuration. To remove the expandable member 200 from the shaft assembly 204, the loading tool 202 may be inserted over the expandable member 200 and the distal portion 206 of the shaft assembly 204 attached thereto. A clinician may then squeeze, pinch, or otherwise urge the tabs 224a, 224b inward, toward the body, sidewall, or coupling portion 210 of the loading tool 202. Squeezing of the tabs 224a, 224b may specifically disengage the curved expandable portions 220a, 220b (or wings), thereby allowing release of the expandable member 200 as a whole. In operation, a user may pinch the tabs 224a, 224b while removing the shaft assembly 204 from the loading tool 202 to release the expandable portions 220a, 220b. The loading tool 202 may be directionally oriented, i.e., it may have a top, bottom, left, right, etc. The “top” side of the loading tool 202 may be labeled, as may one or more additional features.

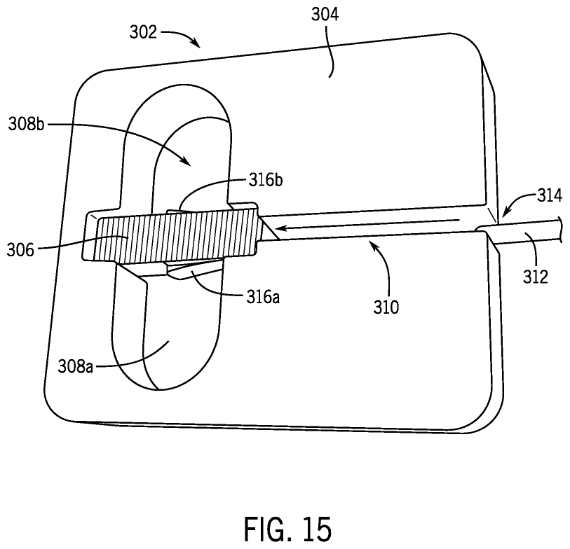

Embodiments of the loading tool 202 may include a handheld component, such as tab 212, or the loading tool 202 may be part of or embedded within a larger body, e.g., a tray member. A tray-embedded loading tool is shown in FIG. 15. Tray-based embodiments may be preferred if additional stability during the expandable member attachment/detachment process is desired, as the loading tool tray 302 may be placed on a flat, stationary surface. As shown in FIG. 15, examples of the loading tool tray 302 may feature a body portion 304, a loading tool component 306 flanked by cavities 308a, 308b configured to accommodate a clinician's fingers, and an elongate channel 310 configured to receive and guide a shaft assembly 312, starting with the shaft assembly's distal end 314, toward and into the open end of the loading tool component 306 (in the direction shown by the arrow). Like loading tool 202, the loading tool component 306 may include disengagement features in the form of tabs 316a, 316b configured to facilitate removal of an expandable member from the distal end 314 of the shaft assembly 312 in response to a user pinching or squeezing the tabs 316a, 316b toward each other. The cavities 308a, 308b provide space for a user to access and pinch the tabs 316a, 316b. The loading tool component 306 may be removable from the tray body 304 in some embodiments. The loading tool component 306 may be fixed within the tray body 304 in other embodiments.

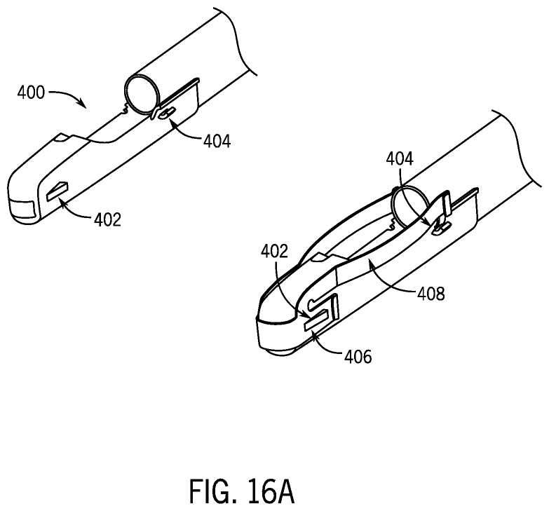

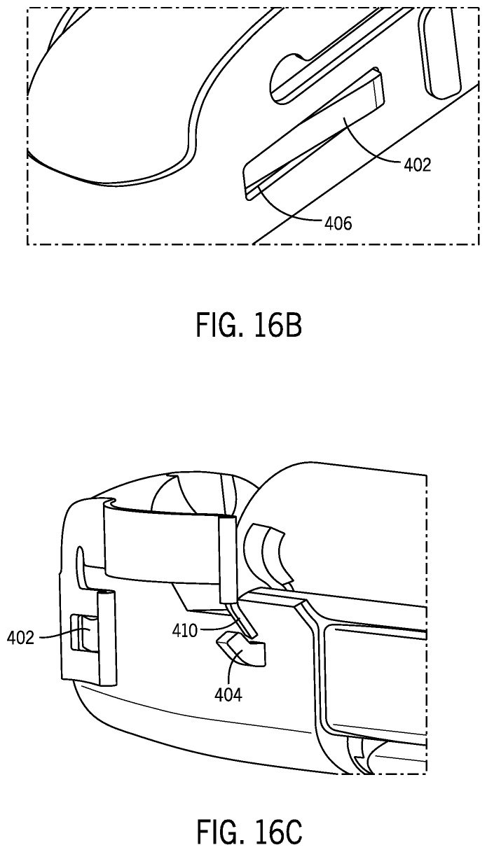

Embodiments may include one or more mechanical engagement features configured to couple and secure an expandable member to the distal end of a delivery shaft assembly. As shown in FIGS. 16A, 16B, and 16C, for example, the distal end portion 400 of a shaft assembly may include at least two engagement portions, for instance in the form of at least one distal protrusion 402 and at least one proximal protrusion 404. The distal protrusion 402 may comprise a ramp portion, surface, or feature, and the proximal protrusion 404 may comprise an angled tab or curved hook portion. The distal protrusion 402 may be configured to be received within a complementary window or cutout 406 of an expandable member 408. The proximal protrusion 404 may be configured to engage or mate with an angled, protruding tab 410 of the expandable member 408.

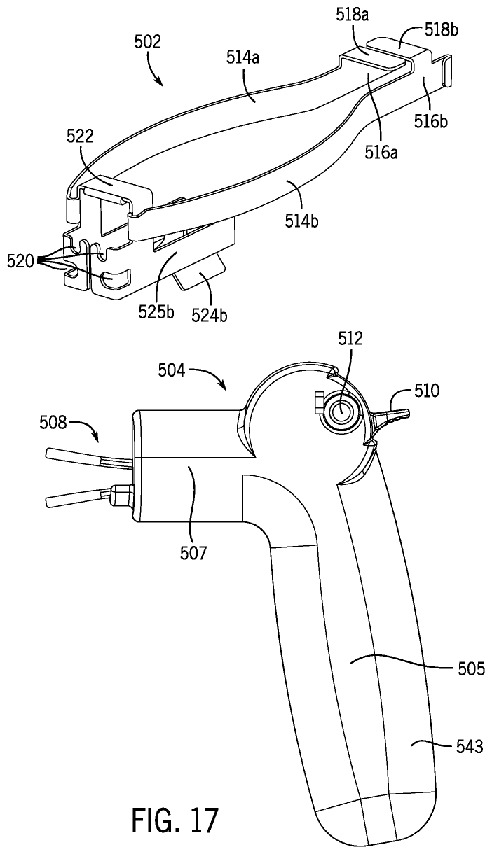

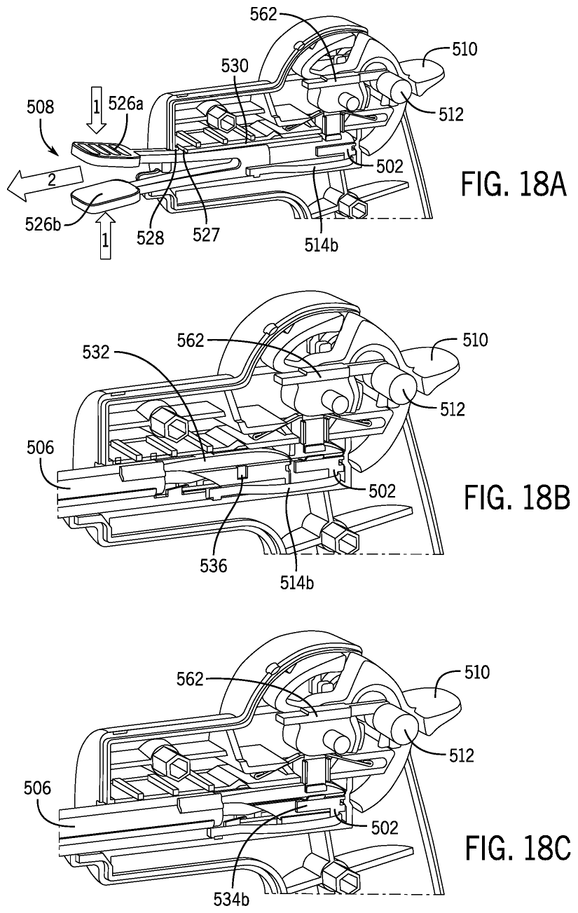

FIG. 17 illustrates another embodiment of a tissue engagement assembly, including a tissue engagement structure in the form of expandable member 502 and an associated handheld loading tool in the form of loading handle assembly 504. Expandable member 502 can be coupled, stored, pre-installed, housed, and/or otherwise positioned within the loading handle assembly 504, the latter configured to facilitate attachment and detachment of the expandable member 502 to and from the distal end or tip of an elongate shaft assembly 506 (see FIG. 18B) of a delivery device (e.g., treatment device 100). The loading handle assembly 504 may be held and manipulated by a user in a manner similar to the delivery device 100 used to deploy prostatic implants, and may similarly feature a gripping portion 505 and a barrel portion 507.

The loading handle assembly 504 may include a removable plug member 508 configured to cover, protect, and stabilize the expandable member 502 within the handle assembly 504 before deployment of the expandable member 502, for example during shipping and handling, so that the expandable member 502 is not damaged, deformed, or contaminated. As further shown in FIGS. 18A-18C, the expandable member 502 is loaded onto the elongate shaft assembly 506 by first removing the plug member 508 from the loading handle assembly 504 (FIG. 18A) and then inserting the distal end of the shaft assembly 506 into the loading handle assembly 504 (FIG. 18B), where the distal end of the shaft assembly 506 attaches to the expandable member 502 positioned therein. The loading handle assembly 504 includes manually engageable actuators, including a release member 510 and a safety lock member 512 together configured to enable controlled release of the expandable member 502 from the shaft assembly 506 in response to user engagement.

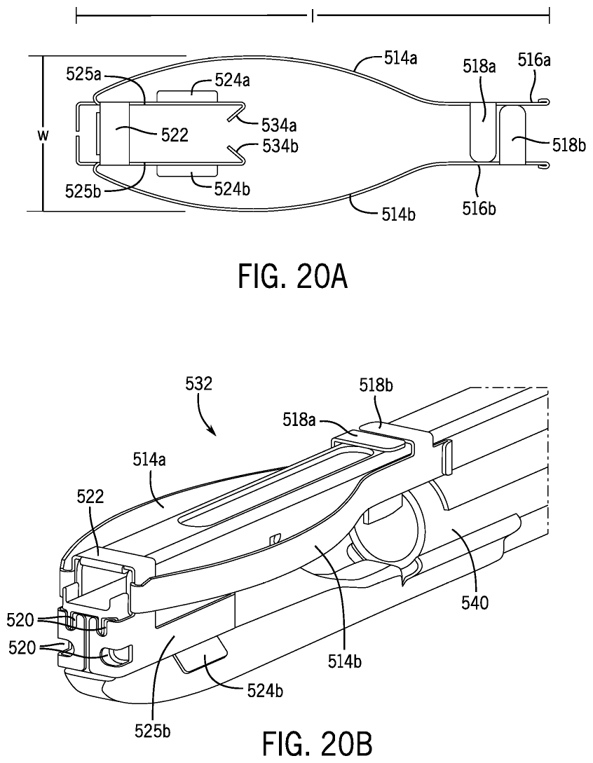

Like expandable member 200, expandable member 502 may include expandable and non-expandable portions. The expandable member 502 may include two expandable curved portions 514a, 514b or “wings” configured to flex such that when deployed within the urethra (and outside any delivery sheath(s)), each expandable portion flexes outwardly, away from the longitudinal axis of the expandable member 502 and into contact with the adjacent tissue, thereby anchoring the expandable member 502 and shaft assembly 506 as a whole at a target site, e.g., within the prostatic urethra, where the expandable member 502 may also improve visibility of the implantation process. The expandable member 502 also includes two arms or proximal end segments 516a, 516b situated in parallel (or substantially in parallel), along with two proximal crossbar members 518a, 518b configured to stabilize the proximal end segments 516a, 516b against an outer surface of the shaft assembly 506. At or near its distal end, the expandable member 502 includes one or more alignment features 520 comprising windows, slots and/or cutout portions configured to align and mate with the distal tip of the shaft assembly 506. A distal crossbar member 522 further stabilizes attachment of the expandable member 502 to the shaft assembly 506. One or more removal tabs 524a, 524b (see also FIG. 20A) flare outward at an angle from two distal arm members 525a, 525b of the expandable member 502. The removal tabs 524a, 524b are configured to disengage the expandable member 502 from the shaft assembly 506 in response to user engagement with the release member 510.

An example process for attaching the expandable member 502 to the distal end of a shaft assembly 506 is depicted in FIGS. 18A-18C. As shown in FIG. 18A, the process may begin by first squeezing two engageable tabs 526a, 526b of the plug member 508 toward each other (“1” arrows). This disengages a projection 527 of the plug member 508 from an abutting edge portion 528 within the handle assembly 504 that otherwise prevents the plug member 508 from being removed. The plug member 508 can then be pulled (“2” arrow) from the inner receiving or holding cavity 530 spanning at least a portion of the barrel portion 507 of the loading handle assembly 504. The distal end 532 of the shaft assembly 506 can then be inserted (e.g., slid) into the empty holding cavity 530, toward the expandable member 502 positioned therein, as shown in FIG. 18B. The user may continue to slide the shaft assembly 506 into the holding cavity 530 until one or more structures of the shaft assembly 506 mate with (e.g., catch) complementary structures of the expandable member 502, for example via snap-fit connection. As shown in FIG. 18C, insertion of the shaft assembly 506 may continue until two barbs or hook portions 534a, 534b (see also FIG. 20A) of the expandable member 502 latch onto one or more complementary recesses 536 of the shaft assembly 506. The shaft assembly 506, now secured to the expandable member 502, may then be removed from the holding cavity 530 of the loading handle assembly 504 and inserted into the urethra of a subject, for example through a sheath previously placed therein (e.g., sheath 28). Attachment of the expandable member 502 to the distal end 532 of the shaft assembly 506 may thus be achieved via a positive mechanical latch mechanism actuated simply by axial insertion of the distal end 532 of the shaft assembly 506 into the loading handle assembly 504, without a separate user step. The attachment mechanism for coupling the expandable member 502 to the shaft assembly 506 may vary, with additional non-limiting examples including one or more recesses, shoulders, notches, catches, ramped surfaces, pawls and teeth, hooks, lips, edges, and/or flanges.

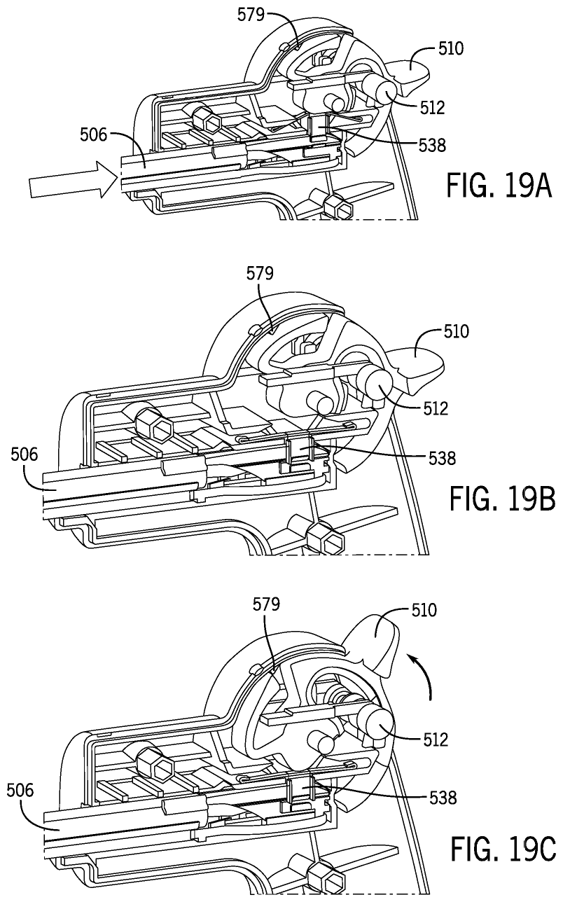

A process for detaching the expandable member 502 from the shaft assembly 506 is shown in FIGS. 19A-19C. As shown in FIG. 19A, the shaft assembly 506 and expandable member 502 attached thereto can be re-inserted (in the direction of arrow) into the holding cavity 530 of the handle assembly 504 until the distal end 532 of the shaft assembly 506 reaches the innermost end of the holding cavity 530. One or more user interactions with the loading handle assembly 504 may then be required to disengage and capture the expandable member 502. For instance, the user may actuate the safety lock member 512, for example by pressing the lock member 512 in either direction (into or out of page-see FIG. 19B) in embodiments featuring a push-button lock member, as depicted. Actuating the safety lock member 512 unlocks the release member 510, such that a user may then lift or sweep the release member 510 (see arrow in FIG. 19C). Actuation of the release member 510 in this manner causes a plunger member 538 within the handle assembly 504 to move (e.g., slide downward), such that it latches onto the expandable member 502 and disengages it from the distal end 532 of the shaft assembly 506. The shaft assembly 506 can then be removed from the holding cavity 530 while the expandable member 502 remains therein. In some embodiments, the expandable member 502 may not be re-attachable to the distal end 532 of the shaft assembly 506 after its release. This intentional anti-reattachment feature may incorporate the use of one or more irreversible deformations, frangible keys, and/or lock geometries.

FIG. 20A provides a top plan view of the expandable member 502. Embodiments of the expandable member 502 may be comprised entirely or partially of stainless steel. Additional materials may also be used, non-limiting examples of which may include other metallic compositions, nitinol, and/or various polymers or polymer compositions. Methods of forming the expandable member 502 may involve bending, molding, 3D printing, machining, welding, and/or otherwise forming the source material, e.g., stainless steel, into the desired shape. The dimensions of the expandable member 502 may vary. In some examples, the total length l of the expandable member 502 may range from about 0.5 inches to about 1.5 inches, or any length therebetween, including about 0.6 inches, 0.7 inches, 0.8 inches, 0.9 inches, 1.0 inches, 1.1 inches, 1.2 inches, 1.3 inches, or 1.4 inches. The maximum width w of the expandable member 502, defined by the curved portions 514a, 514b in their expanded resting state, may also vary, ranging in embodiments from about 0.1 inches to about 0.6 inches, or any width therebetween, including about 0.15 inches, 0.2 inches, 0.25 inches, 0.3 inches, 0.35 inches, 0.4 inches, or 0.45 inches. The cross-sectional thickness of one or more portions of the expandable member 502 may also vary, ranging in some embodiments from about 0.003 inches to about 0.01 inches, or any thickness therebetween, including about 0.004 inches, 0.005 inches, 0.006 inches, 0.007 inches, 0.008 inches, or 0.009 inches. The dimensions of the expandable member 502 should not be viewed as limiting.

FIG. 20B shows the expandable member 502 attached to the distal end 532 of the shaft assembly 506, which also includes a scope tube 540 configured to receive one or more visualization instruments, e.g., a cystoscope, along with various additional components and substructures configured to deploy implants into targeted tissue.

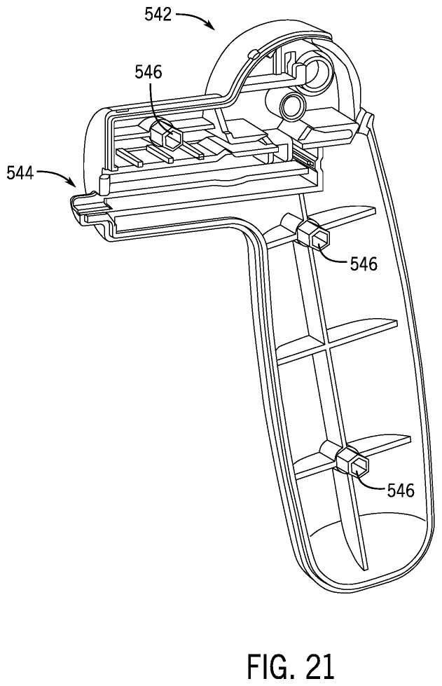

FIG. 21 shows one housing or case component 542 of the handle assembly 504, specifically the right side of the handle assembly, which may be formed with or attached to the housing or case component constituting the left side of the handle assembly, such as left case component 543 (see FIG. 17). The case component 542 includes lead-in lip and key structures 544 configured to guide the distal end 532 of the shaft assembly 506 into the holding cavity 530 in the correct orientation and align the distal end 532 with the expandable member 502. One or more press-fit structures 546 are included to secure the right case component 542 to the left case component 543 to form a housing without the need for additional housing parts. Both case components may be made entirely or at least partially of polycarbonate in some examples, but other materials may be utilized, non-limiting examples of which may include various polymer compositions, including thermoplastics, which may be transparent. Exterior surfaces of the case components may be textured. Methods of forming the case components may involve injection molding, 3D printing, and/or machining, among other techniques.

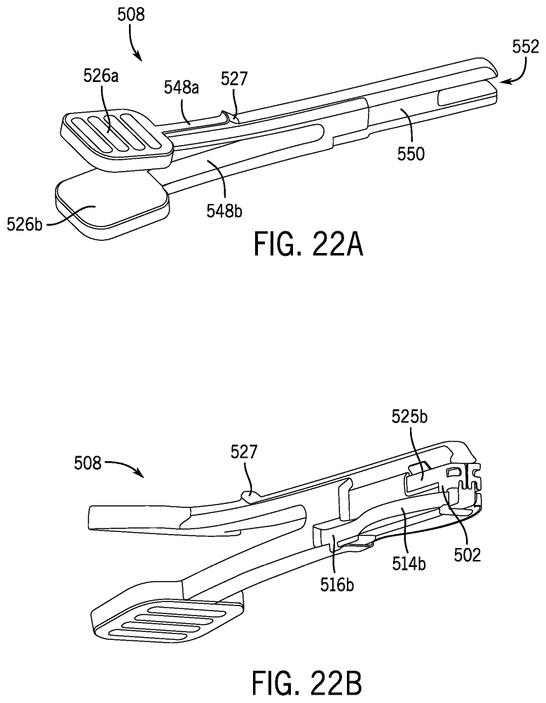

FIG. 22A shows the plug member 508 in isolation, and FIG. 22B shows the plug member 508 coupled with the expandable member 502. The outer surface of the engageable tabs 526a, 526b of the plug member 508 may be textured or patterned to enhance manual gripping. Plug arms 548a, 548b may flex or bend in response to user engagement with the tabs 526a, 526b, e.g., squeezing. A body portion 550 of the plug member 508, positioned entirely or at least partially within the holding cavity 530 of the handle assembly 504 before its removal, may define a cutout 552 configured to receive the distal arm members 525a, 525b of the expandable member 502, further stabilizing the expandable member 502 within the handle assembly 504 and reducing the likelihood of rubbing and wear between the expandable member 502 and the handle assembly 504. Embodiments of the plug member 508 may be made entirely or partially of polycarbonate, but alternative or additional materials may be utilized, non-limiting examples of which may include various polymer compositions, including thermoplastics. Exterior surfaces of the plug member 508 may be textured. Methods of forming the plug member 508 may involve injection molding, 3D printing, and/or machining, among other techniques.

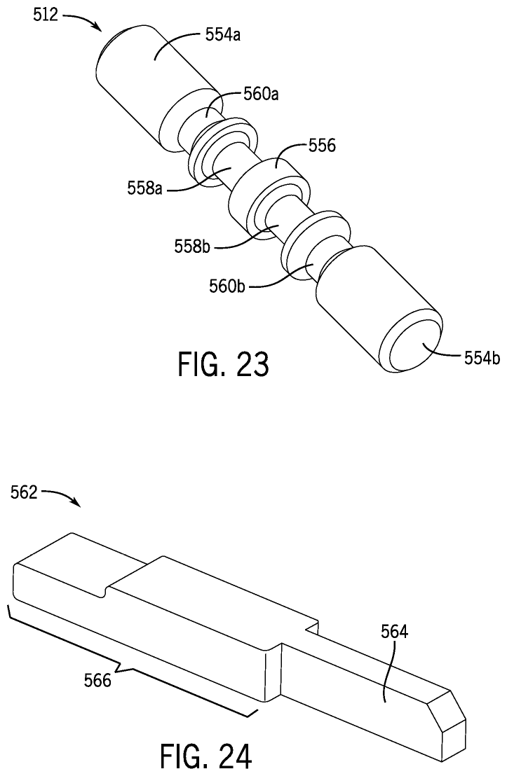

FIG. 23 shows the safety lock member 512, which may include one or more manually engageable portions, which in the illustrated embodiment comprise button members 554a, 554b that may be manually depressed in either direction with respect to the sides of the handle assembly 504. Between the button members 554a, 554b, the body of the safety lock member 512 may have a variable diameter, such that certain segments are configured to selectively interact with certain components of the handle assembly 504. For example, the safety lock member 512 shown in FIG. 23 includes a cylindrical release lever lock portion 556 flanked by narrower slots 558a, 558b, the latter configured to enable rotation of an internal portion of the release member 510, along with one or more latch interfaces 560a, 560b. Together, these features are configured to prevent accidental or unintentional actuation of the release member 510, i.e., prior to actuating the safety lock member 512. Embodiments of the safety lock member 512 may be made entirely or partially of polycarbonate, but alternative or additional materials may be utilized, non-limiting examples of which may include various polymer compositions, including thermoplastics. Exterior surfaces of the safety lock member 512 may be textured. Methods of forming the safety lock member 512 may involve injection molding, 3D printing, and/or machining, among other techniques.

FIG. 24 shows a latch member 562 of the handle assembly 504. The latch member 562 may operate like a spring-board, deflecting out of the way when the safety lock member 512 is being depressed. Once the safety lock member 512 is engaged, the latch member 562 will prevent the safety lock member 512 from moving back to its neutral, resting state position, i.e., prior to user engagement. The illustrated embodiment of the latch member 562 includes a unitary body that defines a narrow extension 564 and a wider step-down portion 566. Some embodiments of the handle assembly 504 may lack the latch member 562. In embodiments that include it, the latch member 562 may be made entirely or partially of polycarbonate, but alternative or additional materials may be utilized, non-limiting examples of which may include various polymer compositions, including thermoplastics. Methods of forming the latch member 562 may involve injection molding, 3D printing, and/or machining, among other techniques.

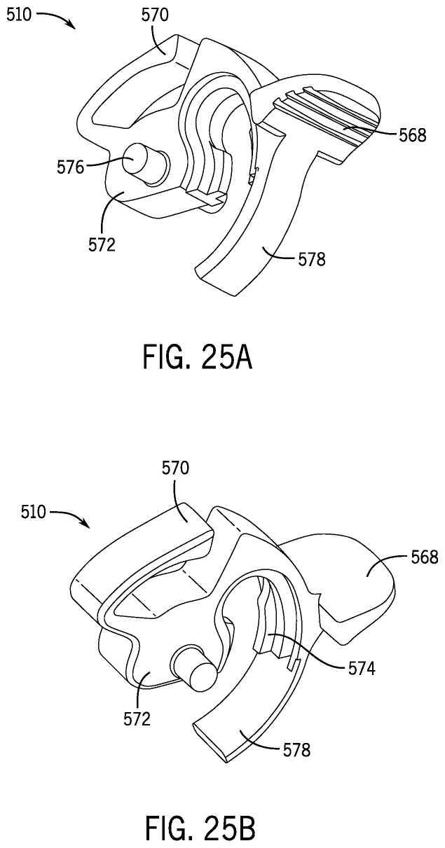

FIGS. 25A and 25B provide different views of the release member 510, at least a portion of which may comprise a rotatable lever. The release member 510 may include a manual engagement portion 568 or surface that protrudes from the interior of the handle assembly 504, extending for instance from the rear of the device. One or more portions of the manual engagement portion 568 may include features configured to prevent slippage when handled by a user, or more specifically the user's thumb. Such features may include a variety of surface patterns comprising one or more grooves, ridges, knurls, hatches, grids, dimples, nubs, bumps, ribs, waves, nodules, or the like. In the example shown, the lower or bottom surface of the engagement portion 568 includes parallel grooves, and the upper or top surface is smooth. The release member 510 may also include a latch portion 570, a cam surface 572, and a safety lock interface 574, each of which may be positioned within the housing defined by the handle assembly 504. A cylindrical protrusion or pin member 576 rotatably engaged with the handle assembly 504 may also be included, along with a curved arm portion 578 configured to further engage the handle assembly 504.

In operation, rotation of the release member 510 may be initiated after manual actuation (e.g., depressing) of the safety lock member 512. Rotation of the release member 510 causes the cam surface 572 to move the plunger member 538, for example by urging or driving the plunger member 538 downward, toward the holding cavity 530. Movement of the plunger member 538 in this manner releases the expandable member 502 from the shaft assembly 506 inserted within the holding cavity 530 of the handle assembly 504. Once the release member 510 is fully rotated or otherwise engaged, the latch portion 570 may engage a retention feature 579 of the case component 542 (see FIGS. 19A-19C), thereby locking the release member 510 in position such that it cannot return or be reset to its original position. This feature of the release member 510 prevents the expandable member 502 from being reattached to the distal end 532 of the shaft assembly 506. The retention mechanism for the release member 510 may vary, with non-limiting examples including one or more recesses, shoulders, notches, catches, ramped surfaces, pawls and teeth, hooks, lips, edges, and/or flanges.

Embodiments of the release member 510 may be made entirely or partially of polycarbonate, but alternative or additional materials may be utilized, non-limiting examples of which may include various polymer compositions, including thermoplastics. Exterior surfaces of the release member 510 may be textured. Methods of forming the release member 510 may involve injection molding, 3D printing, and/or machining, among other techniques.

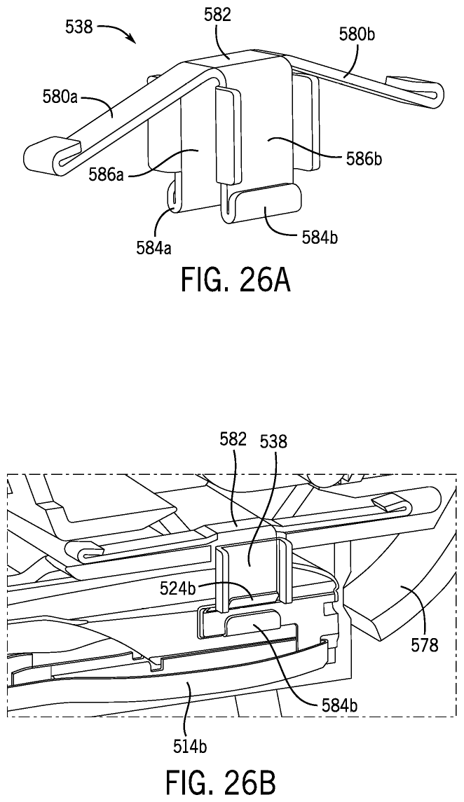

FIG. 26A shows the plunger member 538 in isolation, and FIG. 26B shows the plunger member 538 positioned within the handle assembly 504 after being pressed or urged downward by the cam surface 572 of the release member 510. The plunger member 538 may include two biased arm portions 580a, 580b, a cam contact surface 582, and two hook portions 584a, 584b at the end of two opposing side walls 586a, 586b, respectively. The biased arm portions 580a, 580b may urge the plunger member 538 toward a first, resting state position, which may be above the holding cavity 530 of the handle assembly 504. In this configuration, the arm portions 580a, 580b may define an acute angle with respect to the opposing side walls 586a, 586b. When the release member 510 is engaged, e.g., swept or rotated, the cam surface 572 contacts the cam contact surface 582 and pushes the plunger member 538 downward to a second position, toward the holding cavity 530 and expandable member 502 residing therein. In this configuration, the arm portions 580a, 580b may flex or flatten, such that they are approximately longitudinally aligned along the same plane. Downward movement of the plunger member 538 expands the removal tabs 524a, 524b of the expandable member 502, thereby facilitating or causing the expandable member 502 to disengage from the distal end 532 of the shaft assembly 506. After its detachment from the shaft assembly 506, the expandable member 502 may remain coupled with the plunger member 538, for example by sitting on or against the side walls 586a, 586b and hook portions 584a, 584b.

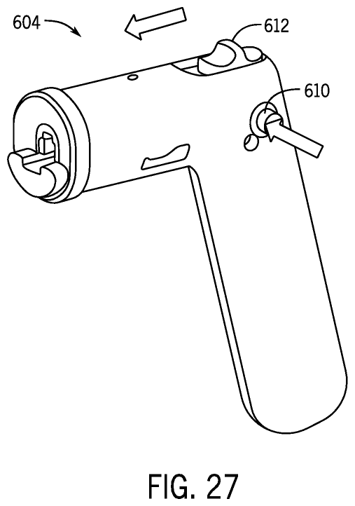

One or more components of the loading handle assembly 504 may vary in different embodiments. For example, the loading handle assembly 604 illustrated in FIG. 27 includes a release member 612 in the form of a slidable button, not a rotatable lever. The safety lock member 610 comprises a push-button, similar to safety lock member 510. Removing an expandable member from the distal end of a shaft assembly in this embodiment involves pressing the safety lock member 610 and sliding the release member 612. The release mechanism may vary across embodiments, which may feature one or more combinations of translatable locking members, slidable engagement features, reciprocating latches, pivotable actuators, trigger-style actuators, toggle actuators, spring clips, cantilevered latches, ball detents, removable locking pins, spring-loading pins, magnetically biased engagements, interference fits, ratcheting mechanisms, and/or elastically deformable locking members, among others.

Forming one or more devices, device components, and device assemblies disclosed herein may involve one or more molding processes, metal-working, machining, and/or multi-part assembly. Plastic components of the various devices can be injection molded in some embodiments. Metal components may be formed via stamping processes. Laser welding may be employed to fix metal components to each other. A variety of connector components, fasteners, and tools, e.g., snaps, screws, etc., may be used to assemble a given device.

A variety of alternative tissue engagement members, components, and devices are also disclosed herein. For instance, rather than attachable/detachable expandable members (or wing portions thereof), embodiments may include an actuatable expandable member attached or fixed to a shaft assembly. Actuatable expandable members may be actuated via controls on the handle of a delivery device. Embodiments of the expandable member may be integral to one or more components or subassemblies of the shaft assembly of a delivery device. Embodiments may also include an expandable member attached to an introducer sheath through which the delivery shaft assembly is inserted during a procedure.

In some examples, the expandable member may comprise an elongate body that extends along all or a portion of the length of the shaft assembly. According to such embodiments, the expandable member may extend the length of the delivery shaft assembly instead of being attached (e.g., clipped) onto the distal end/tip of the shaft assembly. Examples of an elongate tissue engagement structure may be insertable directly into the delivery sheath, after which the delivery shaft assembly may be inserted. Other examples of an elongate tissue engagement structure may be configured such that the elongate delivery shaft is inserted into the elongate tissue engagement structure, after which the coupled components are inserted into the delivery sheath. Embodiments may also feature magnetic attachment mechanisms for attaching the tissue engagement structure to the delivery shaft assembly or portion thereof.

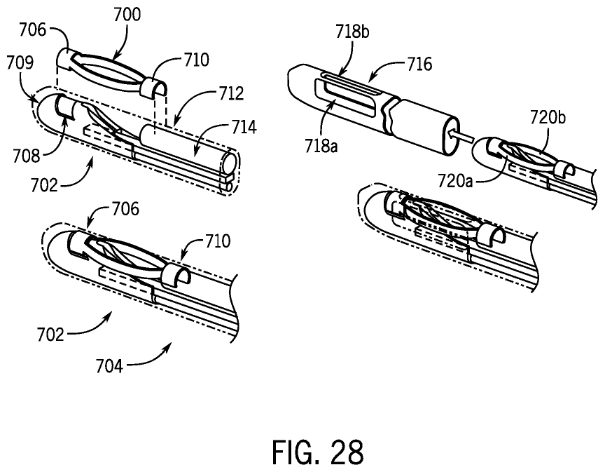

FIG. 28 illustrates an embodiment a tissue engagement structure configured for attachment to and detachment from a shaft assembly of a delivery device. In particular, this embodiment includes a clip-on tissue engagement structure featuring an expandable member 700 configured to clip onto a distal portion 702 of a delivery shaft assembly 704 of a delivery device. The expandable member 700 includes a first, distal clip portion 706 configured to clip onto a complementary groove 708 defined by a distal end-piece 709 of the shaft assembly 704. The opposite end of the expandable member 700 includes a second, proximal clip portion 710 configured to clip onto a distal portion 712 of a scope tube 714 included within the shaft assembly 704, the scope tube configured to receive an imaging device, e.g., endoscope. When coupled, the expandable member 700 and shaft assembly 704 can be inserted into a delivery sheath 716, which includes two cutouts or windows 718a, 718b configured to accommodate lateral extension/expansion of the wings or curved portions 720a, 720b of the expandable member 700.

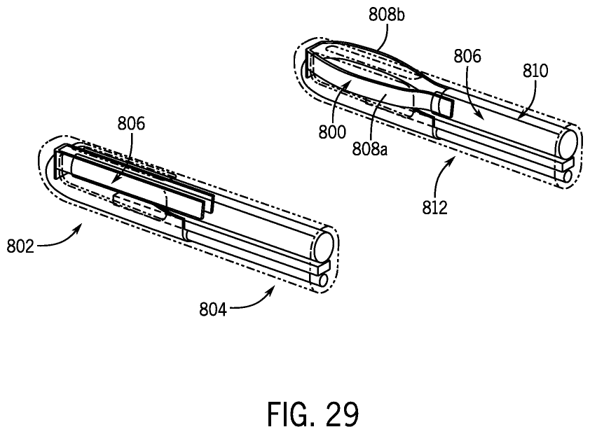

FIG. 29 illustrates a tissue engagement structure featuring slidable compression arms in the shaft assembly of a delivery device. In this embodiment, the expandable member 800 may be fixed to a distal end 802 of the delivery shaft assembly 804. Accordingly, it may not be readily attachable/detachable relative to the shaft assembly. Two or more slidable compression arms 806 within the shaft assembly 804, which together may resemble a movable sleeve component, may compress the curved portions 808a, 808b of the expandable member 800 in response to user engagement at the proximal end of the delivery device, e.g., the handle assembly. In particular, the arms 806 may be slid distally, over the curved portions 808a, 808b of the expandable member 800, to compress or collapse the curved portions 808a, 808b. Proximal retraction of the arms 806 uncovers the curved portions 808a, 808b, allowing them to flex outwardly into the deployed configuration. Proximal movement of the arms 806 may be initiated when the distal end 802 of the delivery shaft assembly reaches a target site within a prostatic urethra, where increased tissue engagement may be needed. A scope tube 810 positioned within the delivery shaft assembly 804, which may be positioned within an introducer sheath 812 during a treatment procedure, may guide the compression arms 806 in both the proximal and distal directions.

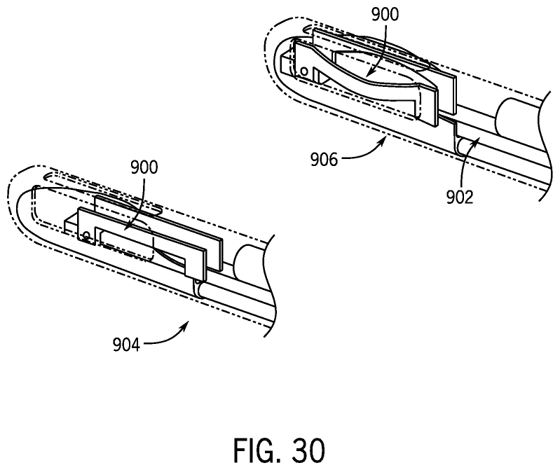

FIG. 30 depicts an embodiment of a tissue engagement structure featuring an expandable member 900 configured to be deployed via movement of an elongate cutter rail member 902 within a delivery shaft assembly 904. The expandable member 900 may be attached or fixed, directly or indirectly, to the cutter rail member 902, which may resemble an elongate rod, tube, or shaft. Like expandable member 800, expandable member 900 may not be readily attachable/detachable relative to the shaft assembly. The cutter rail member 902 constitutes a component of the mechanism used to cut connector portions, e.g., sutures, of implant assemblies during the implant delivery process. The curved portions of the expandable member 900 may be deployed, i.e., allowed to expand or curve outwardly, upon distal advancement of the cutter rail member 902 via a “bail out” feature, which may be activated prior to deployment of the first suture included in a prostatic implant. In the embodiment shown, the expandable member 900 may remain deployed throughout a given procedure and may require retraction of an introducer sheath 906 at the end of the procedure to return to its compressed configuration.

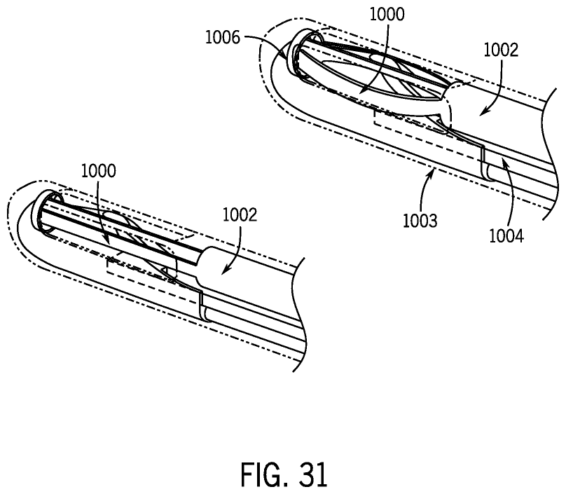

FIG. 31 shows an embodiment of a tissue engagement structure featuring an expandable member coupled or formed with, or constituting a portion or part of, a component of a shaft assembly. In particular, expandable member 1000 constitutes a component or portion of a scope tube 1002 within a shaft assembly 1004. The distal end 1006 of the scope tube 1002 may be fixed to the distal end of the shaft assembly 1004, which may be enclosed within an elongate introducer sheath 1003 during a treatment procedure. Pushing the scope tube 1002 forward (distally) causes the elastic curved portions or wings of the expandable member 1000 to bend or deform into the curved configuration configured for tissue engagement (right image). The curved portions return to the more compact, collapsed configuration when the scope tube 1002 is retracted proximally (left image).

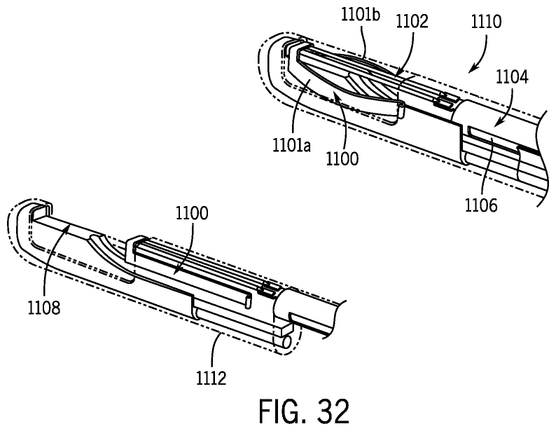

FIG. 32 shows an embodiment of a tissue engagement structure that includes an expandable member 1100 having curved portions 1101a, 1101b attached to a support rod (or rods) 1102, which is attached directly or indirectly to a sleeve member 1104. The sleeve member 1104 may be configured to slide over a scope tube 1106 of the shaft assembly. The sleeve member 1104 may be attached or fixed to the proximal end(s) of the support rod(s) 1102, such that pushing the sleeve member 1104 distally also moves the curved portions 1101a, 1101b of the expandable member 1100 distally, into the open area 1108 defined by the shaft assembly 1110 and surrounding introducer sheath 1112. When the sleeve member 1104 is retracted proximally (left image), the curved portions 1101a, 1101b of the expandable member 1100 are compressed within the sheath 1112. In this embodiment, the open area 1108 for viewing needle deployment may be increased or maximized.

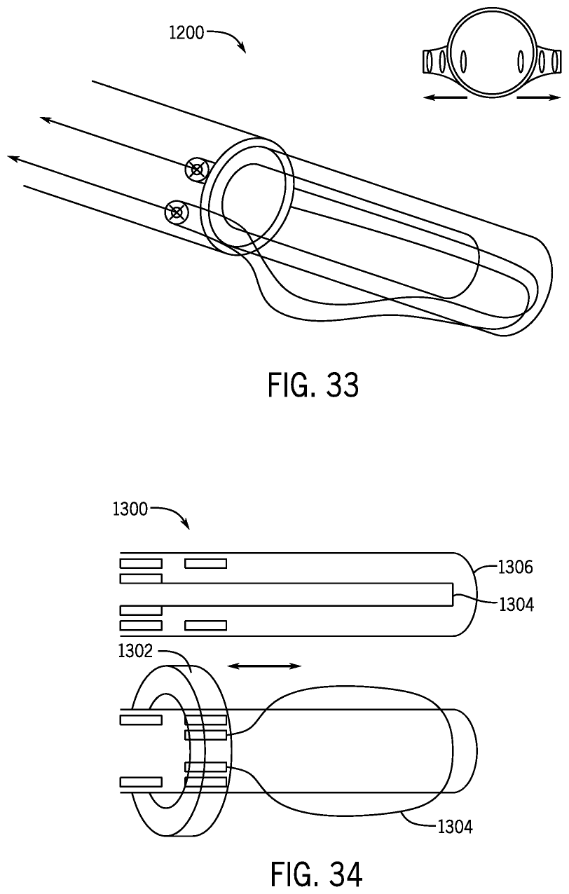

FIG. 33 shows an embodiment of a tissue engagement structure or mechanism 1200 that includes a nitinol expandable member covered in a plastic coating. The nitinol structure may provide enhanced flexibility relative to expandable members comprised of other materials, e.g., metal.

FIG. 34 shows an embodiment of a tissue engagement structure or mechanism 1300 that includes one or more magnetic components 1302 configured to switch an expandable member 1304 between deployed (e.g., expanded) and contracted states. The magnetic component(s) may be separate from and/or coupled to the shaft assembly 1306. In some examples, the magnetic component(s) may comprise a ring-shape component configured to be moved or slid longitudinally along the shaft assembly.

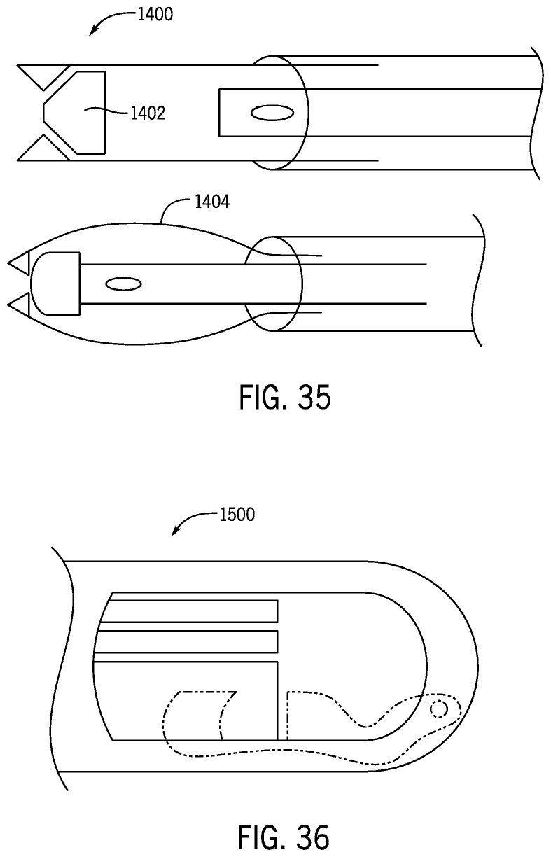

FIG. 35 shows an embodiment of a tissue engagement structure or mechanism 1400 that includes a one-way ratchet 1402 to facilitate expansion and compression of an expandable member 1404.

FIG. 36 shows an embodiment of a tissue engagement structure or mechanism 1500 in which the expandable member is fixed to an introducer sheath, i.e., not the shaft assembly of a delivery device. If or when the expandable member is needed, a sheath that includes the expandable member can be exchanged with a sheath lacking the expandable member.

FIG. 37 shows an embodiment of a tissue engagement structure or mechanism 1600 featuring wire actuation of an expandable member, with a perspective view shown on the right and an end view shown on the left. In this embodiment, the expandable member may comprise one or more wires 1602, e.g., two, three, four, five, six wires or more, configured to slide distally and expand outwardly in the deployed state.

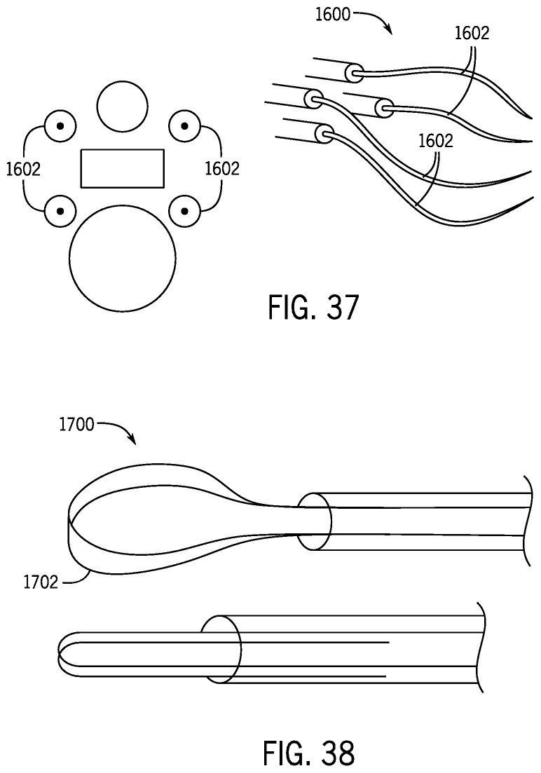

FIG. 38 shows an embodiment of a tissue engagement structure or mechanism 1700 that includes a wire-based expandable member 1702 resembling a whisk. The expandable member 1702 may be wire-or sheet metal-based. The expandable member 1702 may remain concealed within an introducer sheath until pushed distally, at which point it pops or springs open into the deployed state.

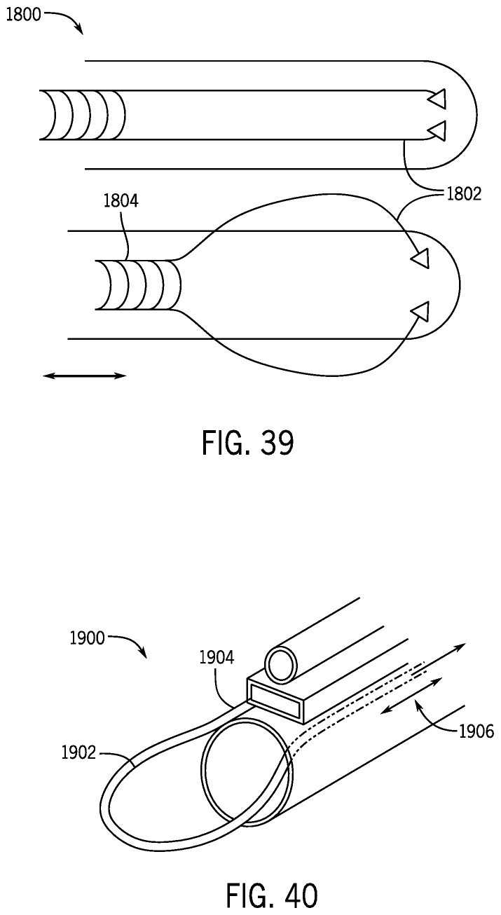

FIG. 39 shows an embodiment of a tissue engagement structure or mechanism 1800 featuring an expandable member 1802 directly or indirectly coupled to a separate component 1804 of a shaft assembly of a delivery device, the separate component comprising one or more rings or push members configured to be pushed or slid distally, thereby advancing and deploying the expandable member, the distal end of which may be fixed at one or more points to the distal end of the shaft assembly.

FIG. 40 shows an embodiment of a tissue engagement structure or mechanism 1900 that includes a wire component 1902 fixed to the shaft assembly at one end 1904 and slidable at the other end 1906. Pushing and pulling the wire component 1902 from the slidable end 1906 causes the wire component to toggle between expanded and contracted configurations.

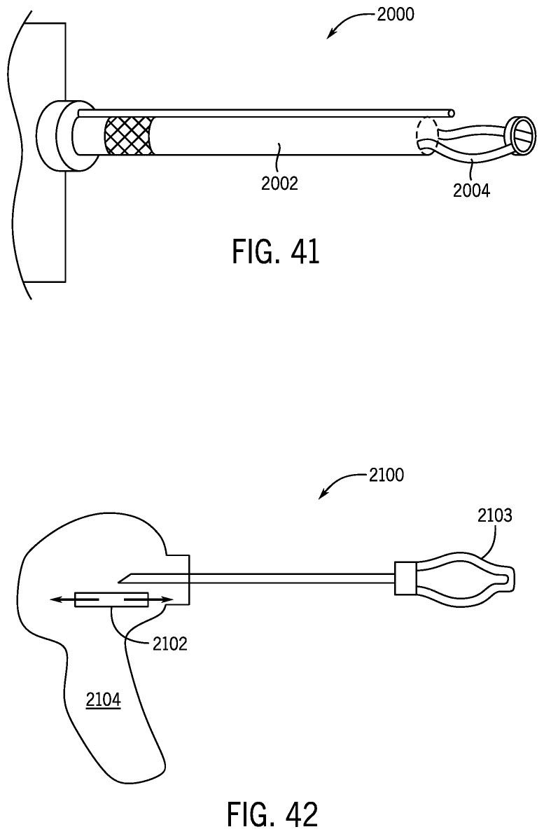

FIG. 41 shows an embodiment of a tissue engagement structure or mechanism 2000 that includes a shaft assembly 2002 configured to actuate an expandable member 2004, the expandable member fixed at its distal end. Longitudinal movement of the shaft assembly 2002 (or component thereof) in the distal direction causes the expandable member 2004 to expand, and longitudinal movement of the shaft assembly 2002 (or component thereof) in the proximal direction causes the expandable member 2004 to contract.

FIG. 42 shows an embodiment of a tissue engagement structure or mechanism 2100 that includes an actuator 2102 or control feature for an expandable member 2103, the actuator positioned on or in the handle assembly 2104 of the delivery system. This embodiment enables user control of the expansion and contraction of the elongate member 2103 via one or more controls or actuators on the handle.

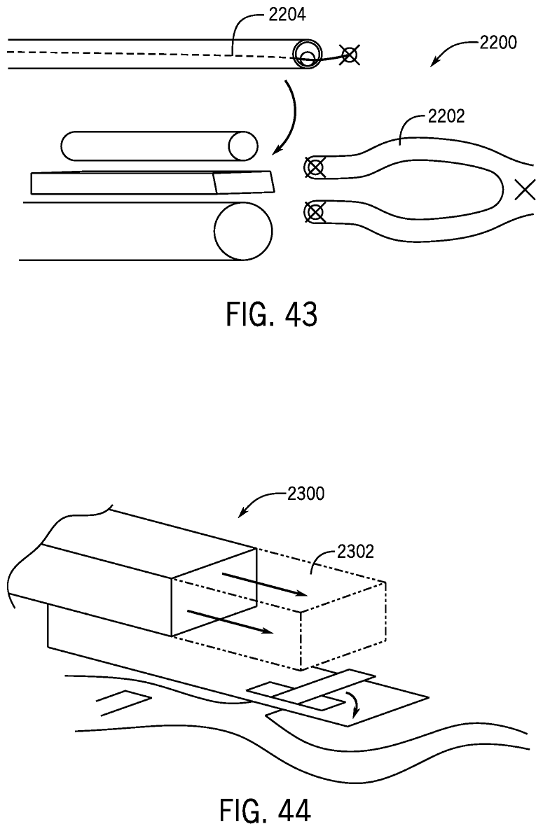

FIG. 43 shows an embodiment of a tissue engagement structure or mechanism 2200 that includes a stamped expandable member 2202 and a wire or cable component 2204 configured to expand and contract the expandable member.

FIG. 44 shows an embodiment of a tissue engagement structure or mechanism 2300 in which sliding of a cutter member 2302 for the implant suture(s) within the shaft assembly causes deployment and contraction of an expandable member.

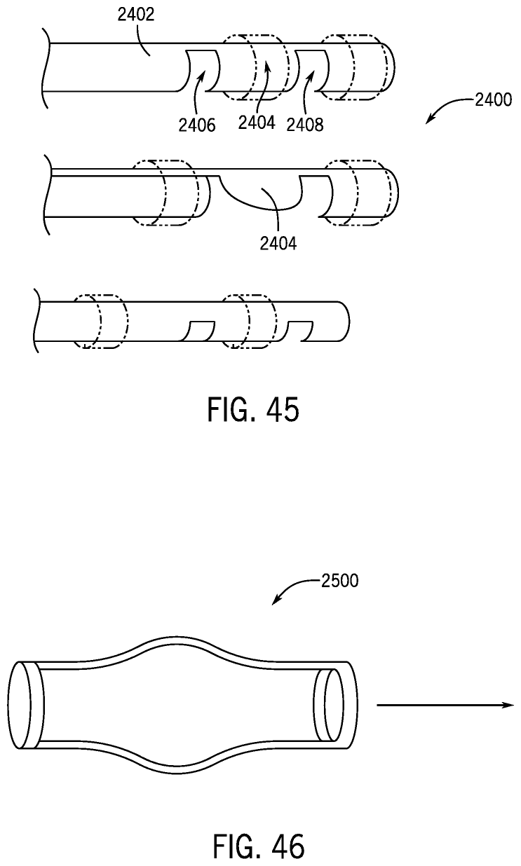

FIG. 45 shows an embodiment of a tissue engagement structure or mechanism 2400 in which the expandable member 2402 comprises a tube (e.g., hypotube), which may be laser-cut. The tube may be cut to include a wide middle portion 2404 flanked by two cutouts 2406 and 2408. The wide middle portion 2404 may expand and contract in response to movement of the expandable member relative to an introducer sheath.

FIG. 46 shows an embodiment of a tissue engagement structure or mechanism 2500 fabricated via cutting or forming a tube, e.g., hypotube, instead of stamping a flat metal piece.

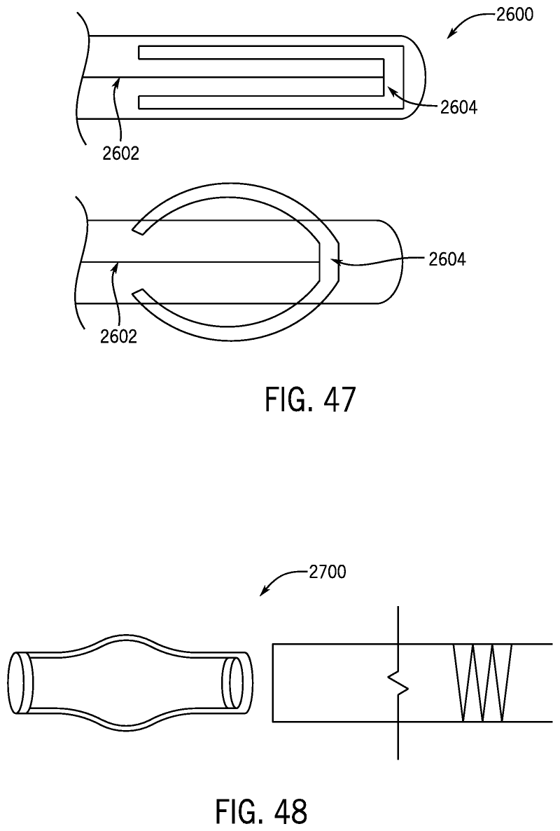

FIG. 47 shows an embodiment of a tissue engagement structure or mechanism 2600 that includes a pull-wire 2602 attached to an expandable member 2604, the pull-wire configured to flex and expand the expandable member by pulling the expandable member in the proximal direction.

FIG. 48 shows an embodiment of a tissue engagement structure or mechanism 2700 that may be activated by an actuator, e.g., knob, configured to transition an expandable member between expanded and contracted states. Embodiments of the actuator may be mounted on the delivery handle of a delivery device.

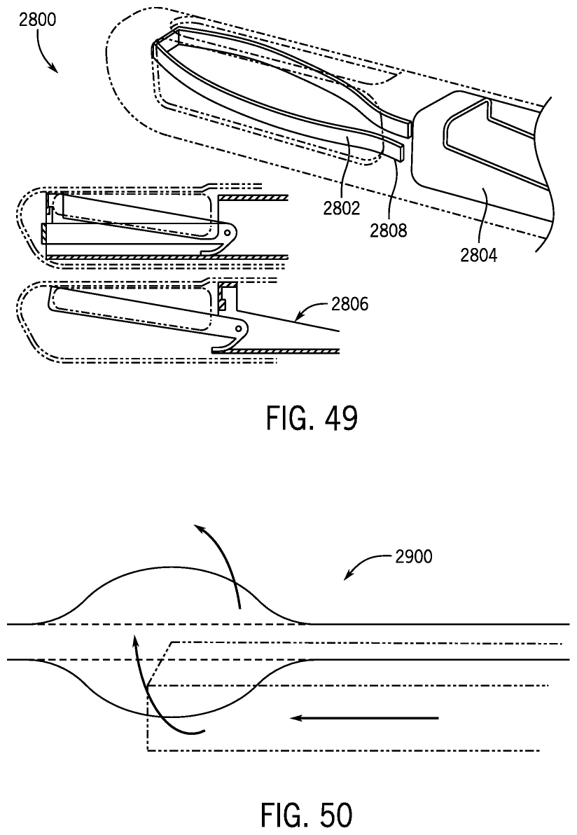

FIG. 49 shows an embodiment of a tissue engagement structure or mechanism 2800 that includes an expandable member 2802 configured to expand/contract and rotate in response to distal/proximal sliding of a sleeve member 2804 positioned within a shaft assembly and having angled sidewalls 2806. The curved portions of the expandable member 2802 may be hinged at their proximal ends 2808.

FIG. 50 shows an embodiment of a tissue engagement structure or mechanism 2900 that includes one or more splayed members. According to this embodiment, the curved portions of the expandable member may be pushed up and outward (relative to the shaft assembly) during expansion.

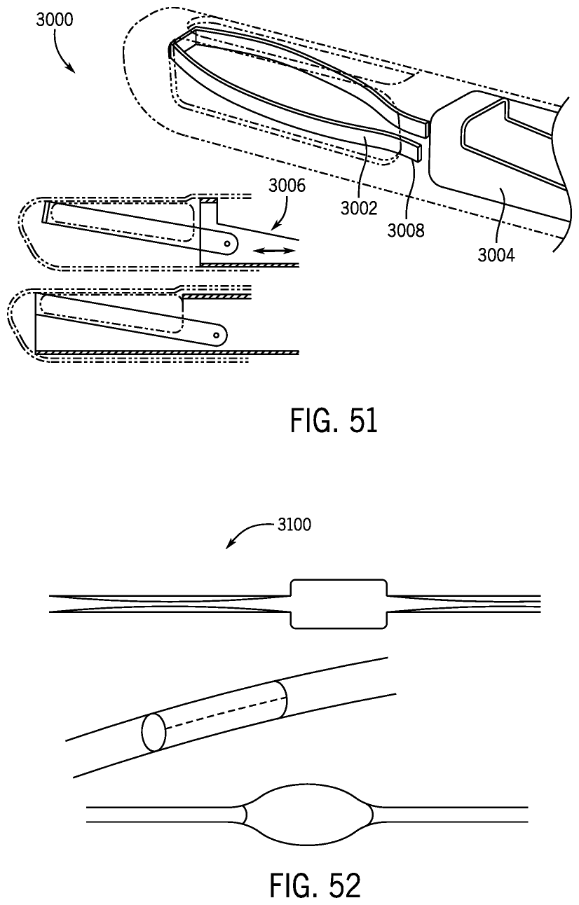

FIG. 51 shows an embodiment of a tissue engagement structure or mechanism 3000 that includes an expandable member 3002 configured to expand/contract in response to distal/proximal sliding of a sleeve member 3004 positioned within the shaft assembly and having angled sidewalls 3006. The curved portions of the expandable member 3002 may be hinged at their proximal ends 3008.

FIG. 52 shows an embodiment of a tissue engagement structure or mechanism 3100 that includes a splayed tube member configured to cause expansion of the tissue engagement structure upon buckling or compressing the tube member.

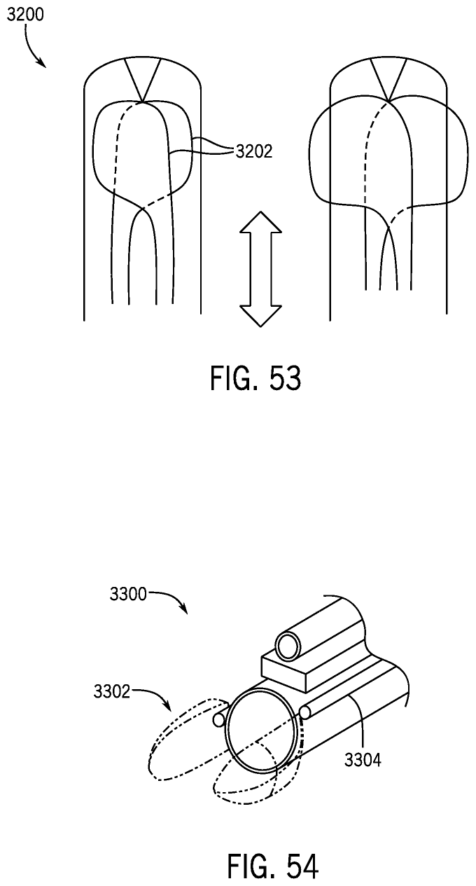

FIG. 53 shows an embodiment of a tissue engagement structure or mechanism 3200 that includes an expandable member comprising two wire components 3202, e.g., loops, that expand outwardly.

FIG. 54 shows an embodiment of a tissue engagement structure or mechanism 3300 that includes an inflatable expandable member 3302. This embodiment may include an elongate inflation tube 3304 in the shaft assembly configured to receive and transmit various inflation agents, e.g., air, saline, etc.

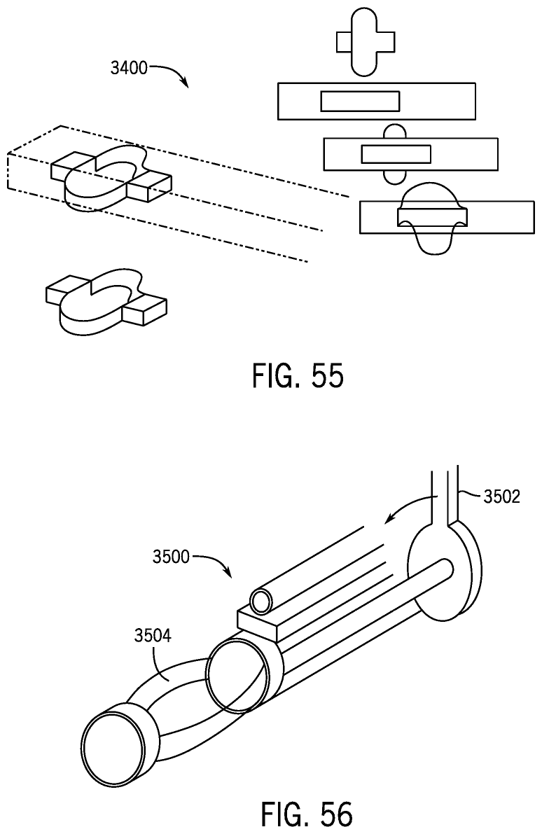

FIG. 55 shows an embodiment of a tissue engagement structure or mechanism 3400 that includes an expandable member inserted into, not clipped onto, a shaft assembly. Insertion of the expandable member is performed outside of the patient, after which the shaft assembly may be inserted back into and through a delivery sheath.

FIG. 56 shows an embodiment of a tissue engagement structure or mechanism 3500 that includes a rotatable lever 3502 configured to drive actuation of an expandable member 3504 in response to user engagement.

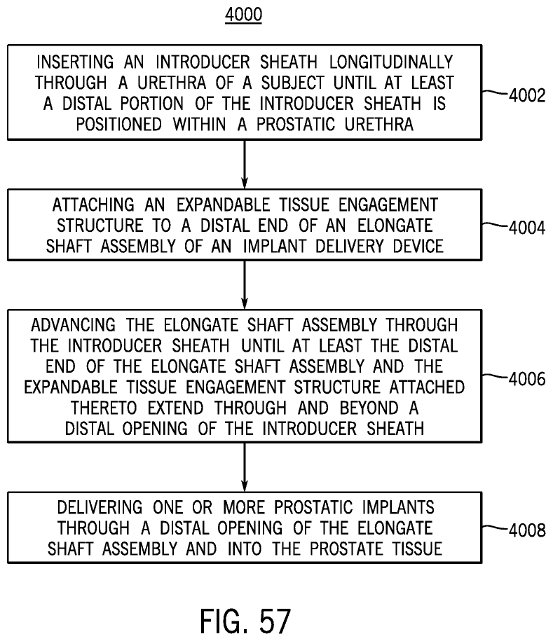

FIG. 57 is a flow diagram of one non-limiting example of a method 4000 of compressing prostate tissue in accordance with embodiments of the present disclosure. The example method 4000 may be performed during a variety of medical procedures, including procedures implemented to treat a subject afflicted with BPH, an enlarged prostate, or one or more symptoms thereof. The method may be implemented by one or more of the disclosed devices, systems, assemblies, and/or components thereof. One or more additional steps may be included, and certain steps may also be omitted or performed in a different sequence than depicted.

In the embodiment shown, the method 4000 begins at step 4002 by “inserting an introducer sheath longitudinally through a urethra of a subject until at least a distal portion of the introducer sheath is positioned within a prostatic urethra.”

At step 4004, the method involves “attaching an expandable tissue engagement structure to a distal end of an elongate shaft assembly of an implant delivery device.” A variety of expandable tissue engagement structures are disclosed herein, including expandable member 200 and 502. In some examples, attaching the expandable tissue engagement structure to the distal end of the elongate shaft assembly comprises inserting the distal end of the elongate shaft assembly into a cavity defined by a handheld loading tool.

At step 4006, the method involves “advancing the elongate shaft assembly through the introducer sheath until at least the distal end of the elongate shaft assembly and the expandable tissue engagement structure attached thereto extend through and beyond a distal opening of the introducer sheath.” The expandable tissue engagement structure may anchor the distal end of the elongate shaft assembly within the prostatic urethra by expanding and pressing against the inner wall of the prostatic urethra. This may prevent or minimize unwanted movement of the shaft assembly during the implant delivery process, as the implants often require precise positioning within the neighboring prostate tissue.

At step 4008, the method involves “delivering one or more prostatic implants through a distal opening of the elongate shaft assembly and into the prostate tissue.” After deployment of the implants, the elongate shaft assembly can be removed from the subject and the tissue engagement structure may be detached from the elongate shaft assembly. In some examples, the prostatic implants compress the prostate tissue as a means of treating benign prostatic hyperplasia.

One or more of the aforementioned steps may be repeated or modified. In some examples, the method may further involve removing the elongate shaft assembly from the introducer sheath and reinserting the distal end of the elongate shaft assembly into the cavity of the handheld loading tool. In some examples, the method may further involve engaging a release mechanism of the handheld loading tool to detach the expandable tissue engagement structure from the distal end of the elongate shaft assembly. In some examples, the method may further involve visualizing at least a portion of the elongate shaft assembly using a cystoscope.

The above Detailed Description includes references to the accompanying drawings, which form a part of the Detailed Description. The Detailed Description should be read with reference to the drawings. The drawings show, by way of illustration, specific embodiments in which the present accessory devices and associated methods can be practiced. These embodiments are also referred to herein as “examples.”

The Detailed Description is intended to be illustrative and not restrictive. For example, the above-described examples (or one or more features or components thereof) can be used in combination with each other. For example, one or more of the disclosed devices and components thereof may be combined. Other embodiments can be used, such as by one of ordinary skill in the art upon reviewing the above Detailed Description. Also, various features or components have been or can be grouped together to streamline the disclosure. This should not be interpreted as intending that an unclaimed disclosed feature is essential to any claim. Rather, inventive subject matter can lie in less than all features of a particular disclosed embodiment. Thus, the following embodiments are hereby disclosed and incorporated into the Detailed Description, with each embodiment standing on its own as a separate embodiment or combinable with any other embodiment:

In an embodiment, a system configured to engage one or more lobes of a prostate gland includes a handle assembly; an elongate shaft assembly attached to the handle assembly and configured for insertion into a urethra of a subject; and a tissue engagement structure configured to be attached to and removed from a distal end of the elongate shaft assembly.

In an embodiment, the tissue engagement structure is expandable, such that it is configured to transition between an expanded state and a collapsed state.

In an embodiment, the tissue engagement structure comprises one or more curved portions or wings configured to flex.

In an embodiment, the system further includes a loading tool configured to be held by a user and couple with the tissue engagement structure.

In an embodiment, the loading tool is configured to attach or facilitate attachment of the tissue engagement structure to the distal end of the elongate shaft assembly.

In an embodiment, the loading tool is configured to detach or facilitate the detachment of the tissue engagement structure from the distal end of the elongate shaft assembly.

In an embodiment, the loading tool includes a body portion that includes or defines an inner cavity configured to receive and retain the tissue engagement structure.

In an embodiment, the loading tool comprises one or more disengagement features configured to facilitate removal of the tissue engagement structure from the elongate shaft assembly.

In an embodiment, the loading tool comprises a loading handle assembly.

In an embodiment, the loading handle assembly comprises a body portion or housing that includes or defines an inner cavity configured to receive a distal portion of the shaft assembly.

In an embodiment, the loading handle assembly further comprises a removable plug member configured to cover the tissue engagement structure when the tissue engagement structure is positioned within the inner cavity.

In an embodiment, the loading handle assembly further comprises a release mechanism configured to disengage the tissue engagement structure from the elongate shaft assembly upon manual actuation.

In an embodiment, after manual actuation of the release mechanism, the tissue engagement structure cannot be reattached to the elongate shaft assembly.

In an embodiment, the release mechanism comprises a safety lock member and a rotatable lever member.

In an embodiment, the safety lock member comprises a push-button.

In an embodiment, manual depression of the safety lock member enables manual rotation of the rotatable lever member.

In an embodiment, a loading tool device configured to couple with a tissue engagement structure includes a handheld assembly defining an inner cavity configured to house the tissue engagement structure and receive at least a distal portion of a shaft assembly; and one or more manually engageable release members configured to detach the tissue engagement structure from the distal portion of the shaft assembly.

In an embodiment, the handheld assembly is configured to enable attachment of the tissue engagement structure to the distal portion of the shaft assembly.

In an embodiment, the handheld assembly comprises a handle assembly comprising a grip portion and a barrel portion, wherein the inner cavity is within the barrel portion.

In an embodiment, the handle assembly further comprises a release mechanism configured to disengage the tissue engagement structure from the elongate shaft assembly upon manual actuation.

In an embodiment, the release mechanism comprises a pushable safety lock member and a rotatable lever member.

In an embodiment, the inner cavity is further configured to receive at least a portion of a removable plug member, the removable plug member configured to protect and retain the tissue engagement structure within the inner cavity.

In an embodiment, a method for compressing prostate tissue involves inserting an introducer sheath longitudinally through a urethra of a subject until at least a distal portion of the introducer sheath is positioned within a prostatic urethra; attaching an expandable tissue engagement structure to a distal end of an elongate shaft assembly of an implant delivery device; advancing the elongate shaft assembly through the introducer sheath until at least the distal end of the elongate shaft assembly and the expandable tissue engagement structure attached thereto extend through and beyond a distal opening of the introducer sheath; and delivering one or more prostatic implants through a distal opening of the elongate shaft assembly and into the prostate tissue.

In an embodiment, the expandable tissue engagement structure comprises curved portions configured to transition from a collapsed state to an expanded state when released from the introducer sheath.

In an embodiment, the curved portions are biased away from a midline defined by a longitudinal axis of the elongate shaft assembly such that the curved portions are compressed toward the midline when positioned within the introducer sheath and expand away from the midline when positioned outside the introducer sheath.

In an embodiment, the one or more prostatic implants are configured to compress the prostatic tissue and alleviate constriction of the prostatic urethra.

In an embodiment, attaching the expandable tissue engagement structure to the distal end of the elongate shaft assembly comprises inserting the distal end of the elongate shaft assembly into a cavity defined by a handheld loading tool.

In an embodiment, the expandable tissue engagement structure detaches from the handheld loading tool upon inserting the distal end of the elongate shaft assembly into the cavity.

In an embodiment, the method further involves removing the elongate shaft assembly from the introducer sheath and reinserting the distal end of the elongate shaft assembly into the cavity of the handheld loading tool.

In an embodiment, the method further involves engaging a release mechanism of the handheld loading tool to detach the expandable tissue engagement structure from the distal end of the elongate shaft assembly.

In an embodiment, the release mechanism comprises a manually engageable safety lock member and a manually engageable release member.

In an embodiment, the manually engageable safety lock member comprises a push-button.

In an embodiment, the manually engageable release member comprises a rotatable lever member.

In an embodiment, the manually engageable release member comprises a slidable button.

In an embodiment, the method further involves visualizing at least a portion of the elongate shaft assembly using a cystoscope.

In an embodiment, the method further involves compressing the prostate tissue provides a means for treating benign prostatic hyperplasia.

In an embodiment, each of the one or more prostatic implants each comprises a distal anchor component configured to be inserted through the prostate tissue and deposited against an outer surface of a prostatic capsule; a connector member comprising a suture attached to the distal anchor component and configured to extend through the prostate tissue; and a proximal anchor component attachable to the connector member within the prostatic urethra.

Closing Notes

Certain terms are used throughout this patent document to refer to particular features or components. As one skilled in the art appreciates, different people may refer to the same feature or component by different names. This patent document does not intend to distinguish between components or features that differ in name but not in function.

For the following defined terms, certain definitions shall be applied unless a different definition is given elsewhere in this patent document. The terms “a,” “an,” and “the” are used to include one or more than one, independent of any other instances or usages of “at least one” or “one or more.” The term “or” is used to refer to a nonexclusive or, such that “A or B” includes “A but not B,” “B but not A,” and “A and B.” All numeric values are assumed to be modified by the term “about,” whether or not explicitly indicated. The term “about” generally refers to a range of numbers that one of skill in the art would consider functionally equivalent to the recited value (e.g., having the same function or result). In many instances, the term “about” can include numbers that are rounded to the nearest significant figure. The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.). The terms “patient” and “subject” are intended to include mammals, such as for human or veterinary applications. The terms “distal” and “proximal” are used to refer to a position or direction relative to the treating clinician. “Distal” and “distally” refer to a position that is distant from, or in a direction away from, the treating clinician. “Proximal” and “proximally” refer to a position that is near, or in a direction toward, the treating clinician.

All relative, directional, and ordinal references (including top, bottom, side, front, rear, first, second, third, and so forth) are given by way of example to aid the reader's understanding of the examples described herein. They should not be read to be requirements or limitations, particularly as to the position, orientation, or use unless specifically set forth in the claims. Connection references (e.g., attached, coupled, connected, joined, and the like) are to be construed broadly and may include intermediate members between a connection of elements and relative movement between elements. As such, connection references do not necessarily infer that two elements are directly connected and in fixed relation to each other, unless specifically set forth in the claims.

The scope of the invention should be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended; that is, a device, kit or method that includes features or components in addition to those listed after such a term in a claim are still deemed to fall within the scope of that claim. Moreover, in the following claims, the terms “first,” “second” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

What is claimed is:

1. A system configured to engage one or more lobes of a prostate gland, comprising:
a handle assembly;
an elongate shaft assembly attached to the handle assembly and configured for insertion into a urethra of a subject; and
a tissue engagement structure configured to be attached to and removed from a distal end of the elongate shaft assembly.

2. The system of claim 1, wherein the tissue engagement structure is expandable, such that it is configured to transition between an expanded state and a collapsed state.

3. The system of claim 1, further comprising a loading tool configured to be held by a user and couple with the tissue engagement structure.

4. The system of claim 3, wherein the loading tool is configured to attach the tissue engagement structure to the distal end of the elongate shaft assembly.

5. The system of claim 3, wherein the loading tool is configured to detach the tissue engagement structure from the distal end of the elongate shaft assembly.

6. The system of claim 3, wherein the loading tool defines an inner cavity configured to receive and retain the tissue engagement structure.

7. The system of claim 3, wherein the loading tool comprises one or more disengagement features configured to facilitate removal of the tissue engagement structure from the elongate shaft assembly.

8. The system of claim 3, wherein the loading tool comprises a loading handle assembly.

9. The system of claim 8, wherein the loading handle assembly comprises an inner cavity configured to receive a distal portion of the shaft assembly.

10. The system of claim 9, wherein the loading handle assembly further comprises a removable plug member configured to cover the tissue engagement structure when the tissue engagement structure is positioned within the inner cavity.

11. The system of claim 10, wherein the loading handle assembly further comprises a release mechanism configured to disengage the tissue engagement structure from the elongate shaft assembly upon manual actuation.

12. The system of claim 11, wherein after manual actuation of the release mechanism, the tissue engagement structure cannot be reattached to the elongate shaft assembly.

13. The system of claim 11, wherein the release mechanism comprises a safety lock member and a rotatable lever member.

14. The system of claim 13, wherein the safety lock member comprises a push-button.

15. The system of claim 13, wherein manual depression of the safety lock member enables manual rotation of the rotatable lever member.

16. A loading tool device configured to couple with a tissue engagement structure, the loading tool device comprising:
a handheld assembly defining an inner cavity configured to house the tissue engagement structure and receive at least a distal portion of a shaft assembly; and
one or more manually engageable release members configured to detach the tissue engagement structure from the distal portion of the shaft assembly.

17. The loading tool device of claim 16, wherein the handheld assembly is configured to enable attachment of the tissue engagement structure to the distal portion of the shaft assembly.

18. The loading tool device of claim 16, wherein the handheld assembly comprises a handle assembly comprising a grip portion and a barrel portion, wherein the inner cavity is within the barrel portion.

19. The loading tool device of claim 18, wherein the handle assembly further comprises a release mechanism comprising a pushable safety lock member and a rotatable lever member together configured to disengage the tissue engagement structure from the elongate shaft assembly upon manual actuation.

20. The loading tool device of claim 16, wherein the inner cavity is further configured to receive at least a portion of a removable plug member, the removable plug member configured to protect and retain the tissue engagement structure within the inner cavity.
